(19) United States
(12) Patent Application Publication
(10) Pub. No.: US 2025/0339191 A1
(43) Pub. Date: Nov. 6, 2025

(54) EXPANDABLE PROSTHETIC DEVICE

(71) Applicant: MiRus LLC, Marietta, GA (US)

(72) Inventors: Jay Yadav, Atlanta, GA (US); Charles Brenner, Marietta, GA (US); Anthony Pipitone, Marietta, GA (US)

(21) Appl. No.: 19/197,799

(22) Filed: May 2, 2025

Related U.S. Application Data
(63) Continuation-in-part of application No. 29/941,909, filed on May 13, 2024.
(63) Continuation-in-part of application No. 29/942,097, filed on May 14, 2024.
(63) Continuation-in-part of application No. 29/942,419, filed on May 15, 2024.
(63) Continuation-in-part of application No. 29/942,426, filed on May 15, 2024.
(63) Continuation-in-part of application No. 29/942,443, filed on May 15, 2024.
(63) Continuation-in-part of application No. 29/942,447, filed on May 15, 2024.
(60) Provisional application No. 63/641,770, filed on May 2, 2024.

(51) Int. Cl. A61B 17/84; A61B 17/72; A61B 17/80

(52) U.S. Cl. CPC A61B 17/844; A61B 17/7291; A61B 17/8028

(57) ABSTRACT

An expandable prosthetic device used as a prosthesis during surgery. The expandable prosthetic device is configured for use in the extremities of a body such as, but not limited to, use in the expansion of the lateral or medial column of a foot. The expandable prosthetic device includes a drive block, a linkage block, a drive screw, a first endplate, a second endplate, and first and sets of linkages. Rotation of the drive screw causes movement of the linkage block relative to the drive block and/or movement of the first endplate relative to the second endplate.

### REFERENCED APPLICATIONS

The present disclosure claims priory on U.S. Provisional Patent Application Ser. No. 63/641,770 filed May 2, 2024, which is fully incorporated herein by reference.

The present disclosure is a continuation-in-part of United States Design patents application Ser. Nos. 29/941,909 filed May 13, 2024; Ser. No. 29/942,097 filed May 14, 2024; Ser. No. 29/942,419 filed May 15, 2024; Ser. No. 29/942,426 filed May 15, 2024; Ser. No. 29/942,443 filed May 15, 2024; and Ser. No. 29/942,447 filed May 15, 2024, which are all fully incorporated herein by reference.

### FIELD OF DISCLOSURE

The present disclosure is directed to an expandable prosthetic device that can be used as a prosthesis used during surgery. The expandable prosthetic device is configured for use in the extremities of a body such as, but not limited to, use in the expansion of the lateral or medial column of a foot; however, it will be appreciated that the expandable prosthetic device can be used to facilitate in the repair of injuries, deformities and/or disorders in other regions of the body.

### BACKGROUND OF DISCLOSURE

Various surgical procedures and prosthetic devices are known for the repair and/or correction or repair of foot/ankle injuries, disorders and/or deformities. Current reconstructive procedures include intra-operative shaping of autogenous bone tissue or human allograft bone tissue. Other bone grafting procedures include packing a void with a granular and/or putty-like material. Intra-operative shaping is a time-consuming process, and further the bone tissue used has limited size and shaping potential. The alternative of packing with granular and/or putty-like materials may not provide adequate structural support.

In view of the current state of the art of prosthetic devices for use in correction or repair of foot/ankle injuries, disorders and/or deformities, there is a continued need for improved prosthetic devices.

### SUMMARY OF DISCLOSURE

The present disclosure is directed to an expandable prosthetic device that can be used as a prosthesis used during surgery. The expandable prosthetic device is configured for use in the extremities of a body such as, but not limited to, use in the expansion of the lateral or medial column of a foot; however, it will be appreciated that the expandable prosthetic device can be used to facilitate in the repair of injuries, deformities and/or disorders in other regions of the body.

In one non-limiting aspect of the disclosure, the expandable prosthetic device includes a drive block, a linkage block, a drive screw, a first endplate, a second endplate, and a first set of linkages that includes first and second linkages.

In another and/or alternative non-limiting aspect of the disclosure, the drive block optionally at least partially forms or includes a drive block opening, and the linkage block optionally at least partially forms or includes a linkage block opening. The drive screw is rotatably coupled at least partially in the drive block opening or linkage block opening and is threadingly disposed within the other of the linkage block opening or the drive block opening. In one non-limiting embodiment, a) the drive block includes a drive block opening and a head of the drive screw is rotatably coupled in a portion of the drive block opening, b) the head of the drive screw that is located in the drive block opening is not threadedly coupled to the drive block, c) during rotation of the drive screw, the head of the drive screw is able to rotate within the drive block opening, but does move or moves less than 5% (e.g., 0-5% and all values and ranges therebetween) the longitudinal length of the drive block opening, d) the linkage block includes a linkage block opening and at least a portion of the linkage block opening includes threading, e) the body of the drive screw includes threading that is threadedly connected to at least a portion of the threading in the linkage block opening, f) during rotation of the drive screw a portion of the body of the drive screw moves with the linkable block opening along a longitudinal axis of the linkage block opening, and g) during rotation of the drive screw a distance between the drive block opening and the linkage block opening is caused to change. In another non-limiting embodiment, a) the drive block includes a drive block opening and at least a portion of the drive block opening includes threading, b) a head of the drive screw is threadedly coupled to a portion of the threading in the drive block opening, c) during rotation of the drive screw, the head of the drive screw is able to rotate within the drive block opening, and moves with the drive block opening along a longitudinal axis of the drive block opening, d) the linkage block includes a linkage block opening, e) the body of the drive screw is rotatably connected to at least a portion of the linkage block opening, f) during rotation of the drive screw, a portion of the body of the drive screw is able to rotate within the linkage block opening, but does move or moves less than 5% (e.g., 0-5% and all values and ranges therebetween) the longitudinal length of the linkage block opening, and g) during rotation of the drive screw a distance between the drive block opening and the linkage block opening is caused to change. In another non-limiting embodiment, the head of the drive screw and the drive block opening of the drive block are configured such that the proximal end of the head of the drive screw that is located farthest from the body of the drive screw always remains within the drive block opening of the drive block during the full expansion and fully contraction of the expandable prosthetic device. In another non-limiting embodiment, the head of the drive screw and the drive block opening of the drive block are configured such that a) the head of the drive screw includes a rib about a portion of all of the outer circumference of the head and the rib is position in a slot in a portion or all of an inner circumference of the drive block opening so that the head of the drive screw can rotate in drive block open, but not move along the longitudinal length of the drive block opening during the rotation of the drive screw, and/or b) the head of the drive screw includes a slot about a portion of all of the outer circumference of the head and the slot is position in a rib in a portion or all of an inner circumference of the drive block opening so that the head of the drive screw can rotate in drive block open, but not move along the longitudinal length of the drive block opening during the rotation of the drive screw. As can be appreciated, more than one slot/rib arrangement can be used. As can also be appreciated, other or additional arrangements can be used to allow the head of the drive screw to rotate within the drive block opening, and the head of the drive screw not move along the longitudinal length of the drive block opening during the rotation of the drive screw.

In another and/or alternative non-limiting aspect of the disclosure, a first end portion of the first linkage on the first set of linkages is rotatably coupled the linkage block and the second end portion of the first linkage on the first set of linkages engages the first endplate, and a first end portion of the second linkage on the first set of linkages is rotatably coupled the linkage block and the second end portion of the second linkage on the first set of linkages engages the second endplate. In one embodiment, rotation of the drive screw causes movement of the linkage block relative to the drive block and movement of the first endplate relative to the second endplate. In another non-limiting embodiment, the second end portion of the first linkage on the first set of linkages includes a first linkage pin that is used to a) facilitate in the movement of the first endplate when the drive screw is rotated to cause a distance between the drive block opening and the linkage block opening is caused to change, b) facilitates in maintaining the engagement of the second end portion of the first linkage and/or the first linkage pin to the first endplate during movement of the first endplate when the drive screw is rotated to cause a distance between the drive block opening and the linkage block opening is caused to change, c) rotatably engage the second end portion of the first linkage on the first set of linkages to the first endplate, but not rotatably secured and/or attached to the first endplate, and/or d) rotatably attach the second end portion of the first linkage on the first set of linkages to the first endplate. In another non-limiting embodiment, the second end portion of the second linkage on the first set of linkages includes a second linkage pin that is used to a) facilitate in the movement of the second endplate when the drive screw is rotated to cause a distance between the drive block opening and the linkage block opening is caused to change, b) facilitates in maintaining the engagement of the second end portion of the second linkage and/or the second linkage pin to the second endplate during movement of the second endplate when the drive screw is rotated to cause a distance between the drive block opening and the linkage block opening is caused to change, c) rotatably engage the second end portion of the second linkage on the first set of linkages to the second endplate, but not rotatably secured and/or attached to the second endplate, and/or d) rotatably attach the second end portion of the second linkage on the first set of linkages to the second endplate. In one specific arrangement, the second end portion of the first and second linkage on the first set of linkages includes a first linkage pin that is used to a) facilitate in the movement of the first endplate and second endplate respectively when the drive screw is rotated to cause a distance between the drive block opening and the linkage block opening is caused to change, b) facilitate in maintaining the engagement of the second end portion of the first and second linkages and the first and second linkage pins to the first and second endplates respectively during movement of the first and second endplates when the drive screw is rotated to cause a distance between the drive block opening and the linkage block opening is caused to change, c) rotatably engage the second end portion of the first and second linkages on the first set of linkages to the first and second endplates respectively, but not rotatably secured and/or attached to the first and second endplates respectively.

In another and/or alternative non-limiting aspect of the disclosure, the expandable prosthetic device further includes a second set of linkages that includes first and second linkages, and wherein the second set of linkages are positioned on the opposite side of the expandable prosthetic device from the expandable prosthetic device, and the first end portion of the first linkage on the second set of linkages is rotatably coupled the linkage block and the second end portion of the first linkage on the second set of linkages engages the first endplate, and a first end portion of the second linkage on the second set of linkages is rotatably coupled the linkage block and the second end portion of the second linkage on the second set of linkages engages the second endplate. In one non-limiting arrangement, the second set of linkages is configured to the same or similar to the first set of linkages and performs the same or similar function as the first set of linkages during movement of the first and second endplates when the drive screw is rotated to cause a distance between the drive block opening and the linkage block opening is caused to change.

In another and/or alternative non-limiting aspect of the disclosure, the first portion of the first and second linkages of the first and/or second set of linkages are rotatably coupled to the linkage block along the same rotation axis.

In another and/or alternative non-limiting aspect of the disclosure, the first portion of the first and second linkages of the first and/or second set of linkages are rotatably coupled to the linkage block along a different rotation axis.

In another and/or alternative non-limiting aspect of the disclosure, the drive block includes a slot region that is positioned distal to the drive block opening that is configured to receive at least a portion of the linkage block and allows the linkage block to move along the longitudinal axis of the drive block when the drive screw is rotated to cause a distance between the drive block opening and the linkage block opening is caused to change. In one non-limiting arrangement, the drive block includes first and second side slots that are located on each side of the slot region of the drive block, and wherein the first and second slots are configured to engage a portion of the linkage block to facilitated in the movement and guidance of movement of the linkage block within the slot region of the drive block.

In another and/or alternative non-limiting aspect of the disclosure, the linkage block includes a linkage housing and a linkage bar wherein the linkage bar includes end flanges that are configured to slidably move within the side slots of the slot region of the drive block so as to facilitate in the movement and guidance of movement of the linkage block within the slot region of the drive block. In one non-limiting arrangement, the linkage housing is configured to remain mostly (e.g., 60-100% of the linkage housing remains within the slot region of the drive block and all values and ranges therebetween) or fully within the slot region of the drive block as linkage housing moves within the slot region when the drive screw is rotated to cause a distance between the drive block opening and the linkage block opening is caused to change. In one non-limiting arrangement, the linkage bar is configured to remain mostly (e.g., 51-98% of the linkage housing remains within the slot region of the drive block and all values and ranges therebetween) within the slot region of the drive block and at least a portion of the end flanges is positioned within the side slots of the slot region of the drive block as the linkage housing moves within the slot region when the drive screw is rotated to cause a distance between the drive block opening and the linkage block opening is caused to change. In another non-limiting arrangement, the linkage housing is configured to receive at least a portion of the linkage bar. In another non-limiting arrangement, both the linkage housing and the linkage bar include a screw opening and when the linkage bar is positioned in the linkage housing, and wherein the screw openings of the linkage bar and the linkage housing are configured to align such that at least a portion of the drive screw body is positioned through both of the screw openings of the linkage bar and the linkage housing. In another non-limiting arrangement, the screw opening in the linkage bar and/or linkage housing includes threading that is configured to engage threading on the body of the drive screw.

In another and/or alternative non-limiting aspect of the disclosure, the linkage block opening optionally includes threading that is located distally of the head of the drive screw when the head of the drive screw is rotatably secured in drive block opening. The threading in the linkage block opening is configured to receive threading on an insertion tool that is configured to be used to insert the expandable prosthetic device into a treatment area (e.g., foot, ankle, wrist, hand, spine, etc.), and thereafter the insertion tool removed from the expandable prosthetic device. The insertion tool can be optionally configured to engage the head of the drive screw and be used to rotate the head of the drive screw.

In another and/or alternative non-limiting aspect of the disclosure, the distal portions of one or both the first and second endplates are configured to be pivotally connected to one another and/or pivotally connected to the distal portion of the drive block. In one non-limiting arrangement, the distal portions of one or both the first and second endplates are configured to be pivotally connected to one another. In another non-limiting arrangement, the distal portions of one or both the first and second endplates are configured to be pivotally connected to the distal portion of the drive block. In another non-limiting arrangement, the distal portions of one or both the first and second endplates are configured to be pivotally connected to one another, and one of the first and second endplates are configured to be pivotally connected the distal portion of the drive block. In another non-limiting arrangement, the distal portions of one or both the first and second endplates are not pivotally connected to one another, and bother of the first and second endplates are configured to be pivotally connected the distal portion of the drive block.

In another and/or alternative non-limiting aspect of the disclosure, the expandable prosthetic device optionally includes one or more graft windows, cavities and/or slots. The one or more graft windows, cavities and/or slots, when used, are configured to facilitate in bone and/or tissue growth on the expandable prosthetic device after the expandable prosthetic device has been implanted at a treatment site.

In another and/or alternative non-limiting aspect of the disclosure, the expandable prosthetic device optionally includes first and/or second endplates that include a micro-textured surface and/or one or more teeth.

In another and/or alternative non-limiting aspect of the disclosure, the expandable prosthetic device optionally includes first and second endplates that include planar top surfaces that do not lie within the same plane when the expandable prosthetic device is in the fully expanded position. In one non-limiting arrangement, the angle formed by the plane of 50-100% (and all values and ranges therebetween) of the top surface of the first and second endplates is about 10°-60° (and all values and ranges therebetween) when the expandable prosthetic device is in the fully expanded position.

In another and/or alternative non-limiting aspect of the disclosure, the expandable prosthetic device optionally includes first and second endplates that include planar top surfaces that lie within or closely within the same plane when the expandable prosthetic device is in the fully contracted position. In one non-limiting arrangement, the angle formed by the plane of 50-100% (and all values and ranges therebetween) of the top surface of the first and second endplates is about 0°-5° (and all values and ranges therebetween) when the expandable prosthetic device is in the fully contracted position. In one non-limiting arrangement, the angle formed by the plane of 50-100% (and all values and ranges therebetween) of the top surface of the first and/or second endplates relative to the central axis of the drive block is about 0°-5° (and all values and ranges therebetween) when the expandable prosthetic device is in the fully contracted position.

In another and/or alternative non-limiting aspect of the disclosure, one or more or all of the components of the expandable prosthetic device is partially or fully formed of a metal alloy. In one non-limiting embodiment, a portion or all of the one or more or all of the components of the expandable prosthetic device is formed of a metal alloy selected from a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) refractory metal alloy, or 1) metal alloy that includes at least 5 atomic weight percent (awt. %) or atomic percent (awt. %) rhenium (e.g., 5-99 awt. % rhenium and all values and ranges therebetween). As used herein, atomic weight percent (awt. %) or atomic percent (awt. %) are used interchangeably. As defined herein, the weight percentage (wt. %) of an element is the weight of that element measured in the sample divided by the weight of all elements in the sample multiplied by 100. The atomic percentage or atomic weight percent (awt. %) is the number of atoms of that element, at that weight percentage, divided by the total number of atoms in the sample multiplied by 100. The use of the terms weight percentage (wt. %) and atomic percentage or atomic weight percentage (awt. %) are two ways of referring to metallic alloy and its constituents. As defined herein, a stainless-steel alloy (SS alloy) includes 10-28 wt. % (weight percent) chromium, 0-35 wt. % nickel, 0-4 wt. % molybdenum, 0-2 wt. % manganese, 0-0.75 wt. % silicon, 0-0.3 wt. % carbon, 0-5 wt. % titanium, 0-10 wt. % niobium, 0-5 wt. % copper, 0-4 wt. % aluminum, 0-10 wt. % tantalum, 0-1 wt. % Se, 0-2 wt. % vanadium, 0-2 wt. % tungsten, and at least 50 wt. % iron. One non-limiting stainless steel alloy is 316L stainless-steel that includes 17-19 wt. % chromium, 13-15 wt. % nickel, 2-4 wt. % molybdenum, 2 wt. % max manganese, 0.75 wt. % max silicon, 0.03 wt. % max carbon, balance iron. As defined herein, a cobalt-chromium alloy (CoCr alloy) includes 15-32 wt. % chromium, 1-38 wt. % nickel, 2-18 wt. % molybdenum, 0-18 wt. % iron, 0-1 wt. % titanium, 0-0.15 wt. % manganese, 0-0.15 wt. % silver, 0-0.25 wt. % carbon, 0-16 wt. % tungsten, 0-2 wt. % silicon, 0-2 wt. % aluminum, 0-1 wt. % iron, 30-68 wt. % cobalt, 0-0.1 wt. % boron, 0-0.15 wt. % silver, and 0-2 wt. % titanium. One type of cobalt-chromium alloy is MP35N alloy that includes 18-22 wt. % chromium, 32-38 wt. % nickel, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. %, 0-0.1 wt. % boron, 0-0.15 wt. % silver, and balance cobalt. Two other type of cobalt-chromium alloy are Phynox and Elgiloy alloy that include 38-42 wt. % cobalt, 18-22 wt. % chromium, 14-18 wt. % iron, 13-17 wt. % nickel, 6-8 wt. % molybdenum. Another type of cobalt-chromium alloy is L605 alloy that includes 18-22 wt. % chromium, 14-16 wt. % tungsten, 9-11 wt. % nickel, balance cobalt. As defined herein, a titanium-aluminum-vanadium alloy (TiAlV alloy) includes 5.5-6.75 wt. % aluminum, 3.5-4.5 wt. % vanadium, 85-93 wt. % titanium, 0-0.4 wt. % iron, 0-0.2 wt. % carbon. One type of titanium-aluminum-vanadium alloy is Ti-6Al-4V alloy that includes 3.5-4.5 wt. % vanadium, 5.5-6.75 wt. % aluminum, 0.3 wt. % max iron, 0.08 wt. % max carbon, 0.05 wt. % max yttrium, balance titanium. As defined herein, an aluminum alloy includes 80-99 wt. % aluminum, 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1 wt. % nickel, 0-0.2 wt. % vanadium, 0-0.1 wt. % gallium, and 0-7 wt. % copper. As defined herein, a nickel alloy includes 30-98 wt. % nickel, 5-25 wt. % chromium, 0-65 wt. % iron, 0-30 wt. % molybdenum, 0-32 wt. % copper, 0-32 wt. % cobalt, 2-2 wt. % aluminum, 0-6 wt. % tantalum, 0-15 wt. % tungsten, 0-5 wt. % titanium, 0-6 wt. % niobium, 0-3 wt. % silicon. As defined herein, a titanium alloy includes 80-99 wt. % titanium, 0-6 wt. % aluminum, 0-3 wt. % tin, 0-1 wt. % palladium, 0-8 wt. % vanadium, 0-15 wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron. As defined herein, a tungsten alloy includes 85-98 wt. % tungsten, 0-8 wt. % nickel, 0-5 wt. % copper, 0-5 wt. % molybdenum, 0-4 wt. % iron. As defined herein, a molybdenum alloy includes 90-99.5 wt. % molybdenum, 0-1 wt. % nickel, 0-1 wt. % titanium, 0-1 wt. % zirconium, 0-30 wt. % tungsten, 0-2 wt. % hafnium, 0-2 wt. % lanthanum. As defined herein, a copper alloy includes 55-95 wt. % copper, 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 wt. % cobalt, 0-20 wt. % nickel. As defined herein, a beryllium-copper alloy includes 95-98.5 wt. % copper, 1-4 wt. % beryllium, 0-1 wt. % cobalt, and 0-0.5 wt. % silicon. As defined herein, a refractory metal alloy is a metal alloy that includes at least 20 wt. % of one or more of molybdenum, rhenium, niobium, tantalum or tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc. In one non-limiting embodiment, one or more or all of the components of the expandable prosthetic device is partially or fully formed of a metal alloy that includes at least 15 awt. % rhenium so as to improve the ductility and/or tensile strength of the metal alloy as compared to a metal alloy is that absent rhenium. Such improvement in ductility and/or tensile strength due to the inclusion of at least 15 awt. % rhenium in the metal alloy is referred to as the “rhenium effect.” As defined herein, a “rhenium effect” is a) an increase of at least 10% in ductility of the metal alloy caused by the addition of rhenium to the metal alloy, and/or b) an increase of at least 10% in tensile strength of the metal alloy caused by the addition of rhenium to the metal alloy. In another non-limiting embodiment, the first and/or second endplates of the expandable prosthetic device are partially or fully formed of titanium alloy, molybdenum alloy rhenium alloy, or metal alloy that includes at least 5 awt. % rhenium. In another non-limiting embodiment, the drive block, pins, linkage block, drive screw, and/or linkages are partially or fully formed of titanium alloy, molybdenum alloy rhenium alloy, or metal alloy that includes at least 5 awt. % rhenium. The material used to form the different components of the expandable prosthetic device can be the same or different.

In another and/or alternative non-limiting aspect of the disclosure, one or more portions of the outer surface of the expandable prosthetic device can be coated with an enhancement layers. Non-limiting enhancement layers include chromium nitride (CrN), diamond-like carbon (DLC), titanium nitride (TiN), titanium oxynitride or titanium nitride oxide (TiNOₓ), zirconium nitride (ZrN), zirconium oxide (ZrO₂), zirconium oxynitride (ZrNₓO_{y}) [e.g., cubic ZrN:O, cubic ZrO₂:N, tetragonal ZrO₂:N, and monoclinic ZrO₂:N phase coatings], oxyzirconium-nitrogen-carbon (ZrNC), zirconium OxyCarbide (ZrOC), and combinations of such coatings. In one non-limiting embodiment, the one or more enhancement layers are optionally applied to a portion or all of the outer surface of the expandable prosthetic device by use of a physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process. In another non-limiting embodiment, the thickness of the enhancement layer is greater than 1 nanometer (e.g., 2 nanometers to 100 microns and all values and ranges therebetween), and typically 0.1-25 microns, and more typically 0.2-10 microns. In another non-limiting embodiment, the hardness of the enhancement layer can be at least 5 GPa (ASTM C1327-15 or ASTM C1624-05), typically 5-50 GPa (and all values and ranges therebetween), more typically 10-25 GPa, and still more typically 14-24 GPa. In another non-limiting embodiment, the coefficient of friction (COF) of the enhancement layer can be 0.04-0.2 (and all values and ranges therebetween), and typically 0.6-0.15. In another non-limiting embodiment, the wear rate of the enhancement layer can be 0.5×10-7 mm3/N-m to 3×10-7 mm3/N-m (an all values and ranges therebetween), and typically 1.2×10-7 mm3/N-m to 2×10-7 mm3/N-m. In another non-limiting embodiment, the enhancement layer includes no more than 0.1 wt. % nickel, no more than 0.1 wt. % chromium, and/or no more than 0.1 wt. % cobalt. In another non-limiting embodiment, the outer surface of the metal portion of the expandable prosthetic device includes no more than 0.1 wt. % nickel, no more than 0.1 wt. % chromium, and/or no more than 0.1 wt. % cobalt. The adhesion layer, when used to facilitate in adhering the enhancement layer to the expandable prosthetic device, includes no more than 0.1 wt. % nickel, no more than 0.1 wt. % chromium, and/or no more than 0.1 wt. % cobalt. In accordance with another non-limiting embodiment, the chromium nitride (CrN) coating generally includes 40-85 wt. % Cr (and all values and ranges therebetween), 15-60 wt. % N (and all values and ranges therebetween), 0-10 wt. % Re (and all values and ranges therebetween), 0-10 wt. % Si (and all values and ranges therebetween), 0-2 wt. % O (and all values and ranges therebetween), and 0-2 wt. % C (and all values and ranges therebetween). In another non-limiting embodiment, the diamond-Like Carbon (DLC) coating generally includes 60-99.99 wt. % C (and all values and ranges therebetween), 0-2 wt. % N (and all values and ranges therebetween), 0-10 wt. % Re (and all values and ranges therebetween), 0-20 wt. % Si (and all values and ranges therebetween), and 0-2 wt. % O (and all values and ranges therebetween). In another non-limiting embodiment, the ratio of N to O when forming the TiNOₓ coating is generally 1:10 to 10:1 (and all values and ranges therebetween). In another non-limiting embodiment, the enhancement layer composition generally includes 20-85 wt. % Ti (and all values and ranges therebetween), 0.5-35 wt. % N (and all values and ranges therebetween), 0-10 wt. % Re (and all values and ranges therebetween), and 0.5-35 wt. % O (and all values and ranges therebetween). In another non-limiting embodiment, the enhancement layer composition generally includes 35-90 wt. % Zr (and all values and ranges therebetween), 5-25 wt. % N (and all values and ranges therebetween), 0-10 wt. % Re (and all values and ranges therebetween), 0-20 wt. % Si (and all values and ranges therebetween), 0-2 wt. % O (and all values and ranges therebetween), and 0-2 wt. % C (and all values and ranges therebetween). In another non-limiting embodiment, the enhancement layer composition generally includes 35-90 wt. % Zr (and all values and ranges therebetween), 10-35 wt. % O (and all values and ranges therebetween), 0-2 wt. % N (and all values and ranges therebetween), 0-10 wt. % Re (and all values and ranges therebetween), 0-20 wt. % Si (and all values and ranges therebetween), and 0-2 wt. % C (and all values and ranges therebetween). In another non-limiting embodiment, the enhancement layer composition generally includes 40-95 wt. % Zr (and all values and ranges therebetween), 5-25 wt. % O (and all values and ranges therebetween), and 10-40 wt. % C (and all values and ranges therebetween), 0-2 wt. % N (and all values and ranges therebetween), 0-10 wt. % Re (and all values and ranges therebetween), and 0-20 wt. % Si (and all values and ranges therebetween). In another non-limiting embodiment, the enhancement layer composition generally includes 20-85 wt. % Zr (and all values and ranges therebetween), 0.5-35 wt. % N (and all values and ranges therebetween), and 0.5-35 wt. % O (and all values and ranges therebetween). In another non-limiting embodiment, the enhancement layer composition generally includes 40-95 wt. % Zr (and all values and ranges therebetween), 5-40 wt. % N (and all values and ranges therebetween), and 5-40 wt. % C (and all values and ranges therebetween), 0-2 wt. % O (and all values and ranges therebetween), 0-10 wt. % Re (and all values and ranges therebetween), and 0-20 wt. % Si (and all values and ranges therebetween).

In yet another and/or alternative non-limiting aspect of the present disclosure, the expandable prosthetic device can include, contain, and/or be coated with one or more agents that facilitate in the success of the expandable prosthetic device and/or treated area. The term “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. Non-limiting examples of clinical events that can be addressed by one or more agents include, but are not limited to, viral, fungal, and/or bacterial infection; vascular diseases and/or disorders; digestive diseases and/or disorders; reproductive diseases and/or disorders; lymphatic diseases and/or disorders; cancer; implant rejection; pain; nausea; swelling; arthritis; bone diseases and/or disorders; organ failure; immunity diseases and/or disorders; cholesterol problems; blood diseases and/or disorders; lung diseases and/or disorders; heart diseases and/or disorders; brain diseases and/or disorders; neuralgia diseases and/or disorders; kidney diseases and/or disorders; ulcers; liver diseases and/or disorders; intestinal diseases and/or disorders; gallbladder diseases and/or disorders; pancreatic diseases and/or disorders; psychological disorders; respiratory diseases and/or disorders; gland diseases and/or disorders; skin diseases and/or disorders; hearing diseases and/or disorders; oral diseases and/or disorders; nasal diseases and/or disorders; eye diseases and/or disorders; fatigue; genetic diseases and/or disorders; burns; scarring and/or scars; trauma; weight diseases and/or disorders; addiction diseases and/or disorders; hair loss; cramps; muscle spasms; tissue repair; nerve repair; neural regeneration and/or the like. The type and/or amount of agent included in and/or coated on the expandable prosthetic device can vary. When two or more agents are included in and/or coated on the expandable prosthetic device, the amount of two or more agents can be the same or different. The type and/or amount of agent included on, in, and/or in conjunction with expandable prosthetic device are generally selected to address one or more clinical events. Typically, the amount of agent included on, in, and/or used in conjunction with the expandable prosthetic device is about 0.01-100 μg per mm² and/or at least about 0.00001 wt. % of device; however, other amounts can be used. In one non-limiting embodiment of the disclosure, the expandable prosthetic device can be partially or fully coated and/or impregnated with one or more agents to facilitate in the success of a particular medical procedure. The amount of the two of more agents on, in, and/or used in conjunction with the expandable prosthetic device can be the same or different. The one or more agents can be coated on and/or impregnated in the expandable prosthetic device by a variety of mechanisms such as, but not limited to, spraying (e.g., atomizing spray techniques, etc.), flame spray coating, powder deposition, dip coating, flow coating, dip-spin coating, roll coating (direct and reverse), sonication, brushing, plasma deposition, depositing by vapor deposition, MEMS technology, and rotating mold deposition.

In a further and/or alternative non-limiting aspect of the present disclosure, the one or more agents on and/or in the expandable prosthetic device (when used) can be released in a controlled manner so the area to be treated is provided with the desired dosage of agent over a sustained period of time. As can be appreciated, controlled release of one or more agents on the expandable prosthetic device is not always required and/or desirable. As such, one or more of the agents on and/or in the expandable prosthetic device can be uncontrollably released from the expandable prosthetic device during and/or after insertion of the expandable prosthetic device in the treatment area. It can also be appreciated that one or more agents on and/or in the expandable prosthetic device can be controllably released from the expandable prosthetic device and one or more agents on and/or in the expandable prosthetic device can be uncontrollably released from the expandable prosthetic device. It can also be appreciated that one or more agents on and/or in one region of the expandable prosthetic device can be controllably released from the expandable prosthetic device and one or more agents on and/or in the expandable prosthetic device can be uncontrollably released from another region on the expandable prosthetic device. As such, the expandable prosthetic device can be designed such that 1) all the agent on and/or in the expandable prosthetic device is controllably released, 2) some of the agent on and/or in the expandable prosthetic device is controllably released and some of the agent on the expandable prosthetic device is non-controllably released, or 3) none of the agent on and/or in the expandable prosthetic device is controllably released. The expandable prosthetic device can also be designed such that the rate of release of the one or more agents from the expandable prosthetic device is the same or different. The expandable prosthetic device can also be designed such that the rate of release of the one or more agents from one or more regions on the expandable prosthetic device is the same or different. Non-limiting arrangements that can be used to control the release of one or more agents from the expandable prosthetic device include 1) at least partially coating one or more agents with one or more polymers, 2) at least partially incorporating and/or at least partially encapsulating one or more agents into and/or with one or more polymers, and/or 3) inserting one or more agents in pores, passageway, cavities, etc., in the expandable prosthetic device and at least partially coating or covering such pores, passageway, cavities, etc., with one or more polymers. As can be appreciated, other or additional arrangements can be used to control the release of one or more agents from the expandable prosthetic device.

In another and/or alternative non-limiting aspect of the present disclosure, the expandable prosthetic device, when including and/or coated with one or more agents, can include and/or be coated with one or more agents that are the same or different in different regions of the expandable prosthetic device and/or have differing amounts and/or concentrations in differing regions of the expandable prosthetic device. For instance, the expandable prosthetic device can be 1) coated with and/or include one or more biologicals on at least one portion of the expandable prosthetic device and at least another portion of the expandable prosthetic device is not coated with and/or includes agent; 2) coated with and/or include one or more biologicals on at least one portion of the expandable prosthetic device that is different from one or more biologicals on at least another portion of the expandable prosthetic device; and/or 3) coated with and/or include one or more biologicals at a concentration on at least one portion of the expandable prosthetic device that is different from the concentration of one or more biologicals on at least another portion of the expandable prosthetic device; etc.

In still yet another and/or alternative non-limiting aspect of the present disclosure, one or more portions of the expandable prosthetic device can 1) include the same or different agents, 2) include the same or different amount of one or more agents, 3) include the same or different polymer coatings, 4) include the same or different coating thicknesses of one or more polymer coatings, 5) have one or more portions of the expandable prosthetic device controllably release and/or uncontrollably release one or more agents, and/or 6) have one or more portions of the expandable prosthetic device controllably release one or more agents and one or more portions of the expandable prosthetic device uncontrollably release one or more agents.

In yet another and/or alternative non-limiting aspect of the disclosure, the expandable prosthetic device can include a marker material that facilitates enabling the expandable prosthetic device to be properly positioned in a treatment area. The marker material is typically designed to be visible to electromagnetic waves (e.g., x-rays, microwaves, visible light, infrared waves, ultraviolet waves, etc.) and/or sound waves (e.g., ultrasound waves, etc.); magnetic waves (e.g., MRI, etc.). In one non-limiting embodiment, the marker material is visible to x-rays (i.e., radiopaque). The marker material can form all or a portion of the expandable prosthetic device and/or be coated on one or more portions (flaring portion and/or body portion, at ends of expandable prosthetic device, at or near transition of body portion and flaring section, etc.) of the expandable prosthetic device. The location of the marker material can be on one or multiple locations on the expandable prosthetic device. The size of the one or more regions that include the marker material can be the same or different. The marker material can be spaced at defined distances from one another to form ruler-like markings on the expandable prosthetic device to facilitate in the positioning of the expandable prosthetic device in a treatment area.

The expandable prosthetic device can include one or more surface structures (e.g., pore, channel, pit, rib, slot, notch, bump, teeth, needle, well, hole, groove, etc.). These structures can be at least partially formed by MEMS (e.g., micro-machining, etc.) technology and/or other types of technology.

The expandable prosthetic device can include one or more micro-structures (e.g., micro-needle, micro-pore, micro-cylinder, micro-cone, micro-pyramid, micro-tube, micro-parallelopiped, micro-prism, micro-hemisphere, teeth, rib, ridge, ratchet, hinge, zipper, zip-tie like structure, etc.) on the surface of the expandable prosthetic device. As defined herein, a “micro-structure” is a structure that has at least one dimension (e.g., average width, average diameter, average height, average length, average depth, etc.) that is no more than about 2 mm, and typically no more than about 1 mm. As can be appreciated, when the expandable prosthetic device includes one or more surface structures, 1) all the surface structures can be micro-structures, 2) all the surface structures can be non-micro-structures, or 3) a portion of the surface structures can be micro-structures and a portion can be non-micro-structures. Non-limiting examples of structures that can be formed on the expandable prosthetic devices are illustrated in United States Patent Publication Nos. 2004/0093076 and 2004/0093077, which are incorporated herein by reference.

In still yet another and/or alternative non-limiting aspect of the present disclosure, there is provided a near net process for a body or other metal component of the expandable prosthetic device. In one non-limiting embodiment of the disclosure, there is provided a method of powder pressing materials and increasing the strength post sintering by imparting additional cold work. In one non-limiting embodiment, the green part is pressed and then sintered. Thereafter, the sintered part is again pressed to increase its mechanical strength by imparting cold work into the pressed and sintered part. Generally, the temperature during the pressing process after the sintering process is 20-100° C. (and all values and ranges therebetween), typically 20-80° C., and more typically 20-40° C. As defined herein, cold working occurs at a temperature of no more than 150° C. (e.g., 10-150° C. and all values and ranges therebetween). The change in the shape of the repressed post-sintered part needs to be determined so the final part (pressed, sintered and re-pressed) meets the dimensional requirements of the final formed part. For a Mo47.5Re alloy, MoRe alloy, ReW alloy, molybdenum alloy, tungsten alloy, rhenium alloy, other type of refractory metal alloy, or TWIP alloy formed of a high titanium content, a prepress pressure of 1-300 tsi (1 ton per square inch) (and all values and ranges therebetween) can be used followed by a sintering process of at least 1600° C. (e.g., 1600-2600° C. and all values and ranges therebetween) and a post sintering press at a pressure of 1-300 tsi (and all values and ranges therebetween) at a temperature of at least 20° C. (e.g., 20-100° C. and all values and ranges therebetween; 20-40° C., etc.). There is also provided a process of increasing the mechanical strength of a pressed metal part by repressing the post-sintered part to add additional cold work into the material, thereby increasing its mechanical strength. There is also provided a process of powder pressing to a near net or final part using metal powder. In one non-limiting embodiment, the metal powder used to form the near net or final part includes a minimum of 40% rhenium by weight and at least 30% molybdenum, and the remainder can optionally include one or more elements of tungsten, tantalum, zirconium, iridium, titanium, bismuth, and yttrium. In another non-limiting embodiment, the metal powder used to form the near net or final part includes 20-80 wt. % rhenium (and all values and ranges therebetween), 20-80 wt. % molybdenum (and all values and ranges therebetween), and optionally one or more elements of tungsten, tantalum, zirconium, iridium, titanium, bismuth, and yttrium. In another non-limiting embodiment, the metal powder used to form the near net or final part includes tungsten (20-60 wt. % and all values and ranges therebetween), rhenium (20-80 wt. % and all values and ranges therebetween) and one or more other elements 0-5 wt. % (and all values and ranges therebetween). In another non-limiting embodiment, the metal powder used to form the near net or final part includes tungsten (20-80 wt. % and all values and ranges therebetween), rhenium (20-80 wt. % and all values and ranges therebetween), molybdenum (0-15 wt. % and all values and ranges therebetween), and one or more other elements 0-5 wt. % (and all values and ranges therebetween). In another non-limiting embodiment, the metal powder used to form the near net or final part includes tungsten (20-80 wt. % and all values and ranges therebetween), copper (1-30 wt. % and all values and ranges therebetween), and one or more other elements 0-5 wt. % (and all values and ranges therebetween). In another non-limiting embodiment, the metal powder used to form the near net or final part includes a titanium alloy or a cobalt alloy. The ductility of the refractory metal alloy measured as % reduction in area can increase and yield and ultimate tensile strength can increase.

In accordance with another and/or alternative non-limiting aspect of the present disclosure, there is provide a medical device that can be form by one or more manufacturing processes. These manufacturing processes can include, but are not limited to, laser cutting, etching, annealing, drawing, pilgering, electroplating, electro-polishing, machining, plasma coating, 3D printed coatings, 3D printing, chemical vapor deposition, chemical polishing, cleaning, pickling, ion beam deposition or implantation, sputter coating, vacuum deposition, etc. In one non-limiting embodiment, at least a portion or all of the medical device is formed by a 3 D printing process.

In one non-limiting object of the disclosure, there is provided an expandable prosthetic device that can be used as a prosthesis used during surgery.

In another and/or alternative non-limiting object of the disclosure, there is provided an expandable prosthetic device that is configured for use in the extremities of a body such as, but not limited to, use in the expansion of the lateral or medial column of a foot; however, it will be appreciated that the expandable prosthetic device can be used to facilitate in the repair of injuries, deformities and/or disorders in other regions of the body.

In another and/or alternative non-limiting object of the disclosure, there is provided an expandable prosthetic device that includes a drive block, a linkage block, a drive screw, a first endplate, a second endplate, and a first set of linkages that includes first and second linkages.

In another and/or alternative non-limiting object of the disclosure, there is provided an expandable prosthetic device that includes a drive block, a linkage block, a drive screw, a first endplate, a second endplate, and a first set of linkages that includes first and second linkages, and wherein a) the drive block optionally at least partially forms or includes a drive block opening, and the linkage block optionally at least partially forms or includes a linkage block opening, and b) the drive screw is rotatably coupled at least partially in the drive block opening or linkage block opening and is threadingly disposed within the other of the linkage block opening or the drive block opening.

In another and/or alternative non-limiting object of the disclosure, there is provided an expandable prosthetic device that includes a drive block, a linkage block, a drive screw, a first endplate, a second endplate, and a first set of linkages that includes first and second linkages, and wherein a) the drive block includes a drive block opening and a head of the drive screw is rotatably coupled in a portion of the drive block opening, b) the head of the drive screw that is located in the drive block opening is not threadedly coupled to the drive block, c) during rotation of the drive screw, the head of the drive screw is able to rotate within the drive block opening, but does move or moves less than 5% the longitudinal length of the drive block opening, d) the linkage block includes a linkage block opening and at least a portion of the linkage block opening includes threading, e) the body of the drive screw includes threading that is threadedly connected to at least a portion of the threading in the linkage block opening, f) during rotation of the drive screw a portion of the body of the drive screw moves with the linkable block opening along a longitudinal axis of the linkage block opening, and g) during rotation of the drive screw a distance between the drive block opening and the linkage block opening is caused to change.

In another and/or alternative non-limiting object of the disclosure, there is provided an expandable prosthetic device that includes a drive block, a linkage block, a drive screw, a first endplate, a second endplate, and a first set of linkages that includes first and second linkages, and wherein a) the drive block includes a drive block opening and at least a portion of the drive block opening includes threading, b) a head of the drive screw is threadedly coupled to a portion of the threading in the drive block opening, c) during rotation of the drive screw, the head of the drive screw is able to rotate within the drive block opening, and moves with the drive block opening along a longitudinal axis of the drive block opening, d) the linkage block includes a linkage block opening, e) the body of the drive screw is rotatably connected to at least a portion of the linkage block opening, f) during rotation of the drive screw, a portion of the body of the drive screw is able to rotate within the linkage block opening, but does move or moves less than 5% the longitudinal length of the linkage block opening, and g) during rotation of the drive screw a distance between the drive block opening and the linkage block opening is caused to change.

In another and/or alternative non-limiting object of the disclosure, there is provided an expandable prosthetic device that includes a drive block, and a drive screw and wherein the drive block opening of the drive block is configured such that the proximal end of the head of the drive screw that is located farthest from the body of the drive screw always remains within the drive block opening of the drive block during the full expansion and fully contraction of the expandable prosthetic device.

In another and/or alternative non-limiting object of the disclosure, there is provided an expandable prosthetic device that includes a drive block, and a drive screw and wherein a) the head of the screw includes a rib about a portion of all of the outer circumference of the head and the rib is position in a slot in a portion or all of an inner circumference of the drive block opening so that the head of the drive screw can rotate in drive block open, but not move along the longitudinal length of the drive block opening during the rotation of the drive screw, and/or b) the head of the screw includes a slot about a portion of all of the outer circumference of the head and the slot is position in a rib in a portion or all of an inner circumference of the drive block opening so that the head of the drive screw can rotate in drive block open, but not move along the longitudinal length of the drive block opening during the rotation of the drive screw.

In another and/or alternative non-limiting object of the disclosure, a first end portion of the first linkage on the first set of linkages is rotatably coupled the linkage block and the second end portion of the first linkage on the first set of linkages engages the first endplate, and a first end portion of the second linkage on the first set of linkages is rotatably coupled the linkage block and the second end portion of the second linkage on the first set of linkages engages the second endplate.

In another and/or alternative non-limiting object of the disclosure, rotation of the drive screw causes movement of the linkage block relative to the drive block and movement of the first endplate relative to the second endplate.

In another and/or alternative non-limiting object of the disclosure, the second end portion of the first linkage on the first set of linkages includes a first linkage pin that is used to a) facilitate in the movement of the first endplate when the drive screw is rotated to cause a distance between the drive block opening and the linkage block opening is caused to change, b) facilitates in maintaining the engagement of the second end portion of the first linkage and/or the first linkage pin to the first endplate during movement of the first endplate when the drive screw is rotated to cause a distance between the drive block opening and the linkage block opening is caused to change, c) rotatably engage the second end portion of the first linkage on the first set of linkages to the first endplate, but not rotatably secured and/or attached to the first endplate, and/or d) rotatably attach the second end portion of the first linkage on the first set of linkages to the first endplate.

In another and/or alternative non-limiting object of the disclosure, the second end portion of the second linkage on the first set of linkages includes a second linkage pin that is used to a) facilitate in the movement of the second endplate when the drive screw is rotated to cause a distance between the drive block opening and the linkage block opening is caused to change, b) facilitates in maintaining the engagement of the second end portion of the second linkage and/or the second linkage pin to the second endplate during movement of the second endplate when the drive screw is rotated to cause a distance between the drive block opening and the linkage block opening is caused to change, c) rotatably engage the second end portion of the second linkage on the first set of linkages to the second endplate, but not rotatably secured and/or attached to the second endplate, and/or d) rotatably attach the second end portion of the second linkage on the first set of linkages to the second endplate.

In another and/or alternative non-limiting object of the disclosure, the expandable prosthetic device further includes a second set of linkages that includes first and second linkages, and wherein the second set of linkages are positioned on the opposite side of the expandable prosthetic device from the expandable prosthetic device, and the first end portion of the first linkage on the second set of linkages is rotatably coupled the linkage block and the second end portion of the first linkage on the second set of linkages engages the first endplate, and a first end portion of the second linkage on the second set of linkages is rotatably coupled the linkage block and the second end portion of the second linkage on the second set of linkages engages the second endplate.

In another and/or alternative non-limiting object of the disclosure, the first portion of the first and second linkages of the first and/or second set of linkages are rotatably coupled to the linkage block along the same rotation axis.

In another and/or alternative non-limiting object of the disclosure, the drive block includes a slot region that is positioned distal to the drive block opening that is configured to receive at least a portion of the linkage block and allows the linkage block to move along the longitudinal axis of the drive block when the drive screw is rotated to cause a distance between the drive block opening and the linkage block opening is caused to change.

In another and/or alternative non-limiting object of the disclosure, the drive block includes first and second side slots that are located on each side of the slot region of the drive block, and wherein the first and second slots are configured to engage a portion of the linkage block to facilitated in the movement and guidance of movement of the linkage block within the slot region of the drive block.

In another and/or alternative non-limiting object of the disclosure, the linkage block includes a linkage housing and a linkage bar wherein the linkage bar includes end flanges that are configured to slidably move within the side slots of the slot region of the drive block so as to facilitate in the movement and guidance of movement of the linkage block within the slot region of the drive block.

In another and/or alternative non-limiting object of the disclosure, both the linkage housing and the linkage bar include a screw opening and when the linkage bar is positioned in the linkage housing, and wherein the screw openings of the linkage bar and the linkage housing are configured to align such that at least a portion of the drive screw body is positioned through both of the screw openings of the linkage bar and the linkage housing.

In another and/or alternative non-limiting object of the disclosure, the distal portions of one or both the first and second endplates are configured to be pivotally connected to one another and/or pivotally connected to the distal portion of the drive block.

In another and/or alternative non-limiting object of the disclosure, the expandable prosthetic device optionally includes one or more graft windows, cavities and/or slots.

In another and/or alternative non-limiting object of the disclosure, the expandable prosthetic device optionally includes first and/or second endplates that include a micro-textured surface and/or one or more teeth.

In another and/or alternative non-limiting object of the disclosure, the expandable prosthetic device optionally includes first and second endplates that include planar top surfaces that do not lie within the same plane when the expandable prosthetic device is in the fully expanded position.

In another and/or alternative non-limiting object of the disclosure, the expandable prosthetic device optionally includes first and second endplates that include planar top surfaces that lie within or closely within the same plane when the expandable prosthetic device is in the fully contracted position.

In another and/or alternative non-limiting object of the disclosure, one or more or all of the components of the expandable prosthetic device is partially or fully formed of a metal alloy selected from a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) refractory metal alloy, or l) metal alloy that includes at least 5 awt. % rhenium.

In another and/or alternative non-limiting object of the disclosure, one or more portions of the outer surface of the expandable prosthetic device are coated with an enhancement layers such as chromium nitride (CrN), diamond-like carbon (DLC), titanium nitride (TiN), titanium oxynitride or titanium nitride oxide (TiNOₓ), zirconium nitride (ZrN), zirconium oxide (ZrO₂), zirconium oxynitride (ZrNₓO_{y}) [e.g., cubic ZrN:O, cubic ZrO₂:N, tetragonal ZrO₂:N, and monoclinic ZrO₂:N phase coatings], oxyzirconium-nitrogen-carbon (ZrNC), zirconium OxyCarbide (ZrOC), and combinations of such coatings.

In another and/or alternative non-limiting object of the disclosure, the expandable prosthetic device can optionally be partially or fully be coated with and/or include one or more agents.

In another and/or alternative non-limiting object of the disclosure, one or more portions of the expandable prosthetic device can include a marker material that facilitates enabling the expandable prosthetic device to be properly positioned in the treatment area.

In another and/or alternative non-limiting object of the disclosure, the angle formed by the plane of 50-100% of the top surface of the first and second endplates is about 15°-50° when the expandable prosthetic device is in the fully expanded position.

In another and/or alternative non-limiting object of the disclosure, the angle formed by the plane of 50-100% of the top surface of the first and second endplates is about 20°-40° when the expandable prosthetic device is in the fully expanded position.

In another and/or alternative non-limiting object of the disclosure, the angle formed by the plane of 50-100% of the top surface of the first and second endplates is about 0°-5° when the expandable prosthetic device is in the fully contracted position.

In another and/or alternative non-limiting object of the disclosure, the angle formed by the plane of 50-100% of the top surface of the first and second endplates is about 5°-30° when the expandable prosthetic device is in the fully contracted position.

In another and/or alternative non-limiting object of the disclosure, there is a provision of an expandable prosthetic device that comprises: a) a drive block; and wherein the drive block includes a drive block opening; b) a linkage block; and wherein the linkage block includes a linkage housing and a linkage bar; and wherein the linkage bar is at least partially positioned in the linkage housing; c) a drive screw; and wherein the drive screw includes a head and body portion; and wherein the head is rotatably positioned in within the drive block opening; and wherein the body portion is threadingly connected to the linkage block; d) first and second endplates; and wherein the first endplate includes a front portion and a rear portion; and wherein the second endplate includes a front portion and a rear portion; and wherein the rear portion of the first and second endplates are pivotally connected together; and, e) first and second sets of linkages; and wherein the first set of linkage includes first and second linkages; the second set of linkage includes first and second linkages; the first linkage of said first set of linkages engages the linkage bar and the first endplate; the first linkage of the second set of linkages engages the linkage bar and the first endplate; the second linkage of the first set of linkages engages the linkage bar and the second endplate; the second linkage of the second set of linkages engages the linkage bar and the second endplate; the first and second sets of linkages are spaced from one another; and wherein rotation of the drive screw causes I) movement of the linkage block relative to the first and/or second end plates along a longitudinal length of the expandable prosthetic device as the expandable prosthetic device moves from the fully closed or collapsed position to a fully open or expanded position, and II) movement of the first endplate relative to the second endplate as the expandable prosthetic device moves from the fully closed or collapsed position to a fully open or expanded position; and wherein the drive block and linkage block are spaced from one another when the expandable prosthetic device is in a fully closed or collapsed position, and a spacing between the drive block and linkage block is reduced as the expandable prosthetic device moves from the fully closed or collapsed position to a fully open or expanded position.

Other objects, advantages, and novel features of the present invention will become apparent from the following detailed description of the invention when considered in conjunction with the accompanying drawings.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIG. 1 is an isometric view one non-limiting expandable prosthetic device in a fully open or expanded position in accordance with one non-limiting aspect of the present disclosure.

FIG. 2 is a side view of the expandable prosthetic device of FIG. 1.

FIG. 3 is a distal end view of the expandable prosthetic device of FIG. 1.

FIG. 4 is a proximal end view of the expandable prosthetic device of FIG. 1.

FIG. 5 is a top isometric view an expandable prosthetic device of FIG. 1 in the fully closed or collapsed position.

FIG. 6 is a side view of the expandable prosthetic device of FIG. 7.

FIG. 7 is an exploded view of the expandable prosthetic device of FIG. 1.

FIG. 8 is an isometric view another non-limiting expandable prosthetic device in a fully open or expanded position in accordance with one non-limiting aspect of the present disclosure.

FIG. 9 is a side view of the expandable prosthetic device of FIG. 8.

FIG. 10 is a distal end view of the expandable prosthetic device of FIG. 8.

FIG. 11 is a proximal end view of the expandable prosthetic device of FIG. 8.

FIG. 12 is a top isometric view an expandable prosthetic device of FIG. 8 in the fully closed or collapsed position.

FIG. 13 is a bottom isometric view an expandable prosthetic device of FIG. 8 in the fully closed or collapsed position.

FIG. 14 is a side view of the expandable prosthetic device of FIG. 8.

FIG. 15 is an exploded view of the expandable prosthetic device of FIG. 8.

FIG. 16 is an isometric view another non-limiting expandable prosthetic device in a fully open or expanded position in accordance with one non-limiting aspect of the present disclosure.

FIG. 17 is a side view of the expandable prosthetic device of FIG. 16.

FIG. 18 is a distal end view of the expandable prosthetic device of FIG. 16.

FIG. 19 is a proximal end view of the expandable prosthetic device of FIG. 16.

FIG. 20 is a top isometric view an expandable prosthetic device of FIG. 16 in the fully closed or collapsed position.

FIG. 21 is a bottom isometric view an expandable prosthetic device of FIG. 16 in the fully closed or collapsed position.

FIG. 22 is a side view of the expandable prosthetic device of FIG. 16.

FIG. 23 is an exploded view of the expandable prosthetic device of FIG. 16.

### DETAILED DESCRIPTION OF NON-LIMITING EMBODIMENTS

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

The devices, systems, and methods described herein include an expandable prosthetic device 1000. The expandable prosthetic device 1000 includes linkages that allow for movement of the endplates relative to each other.

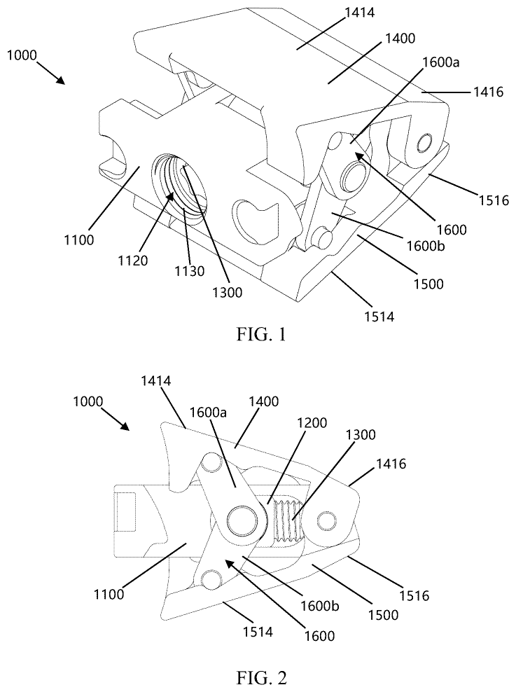

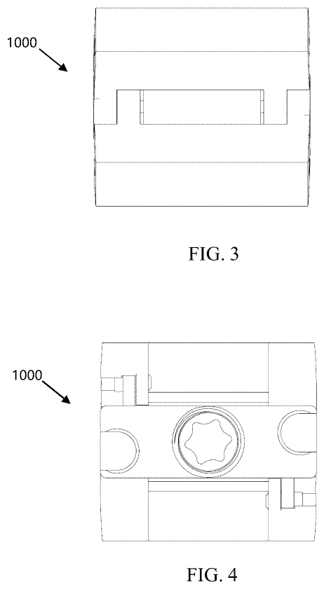

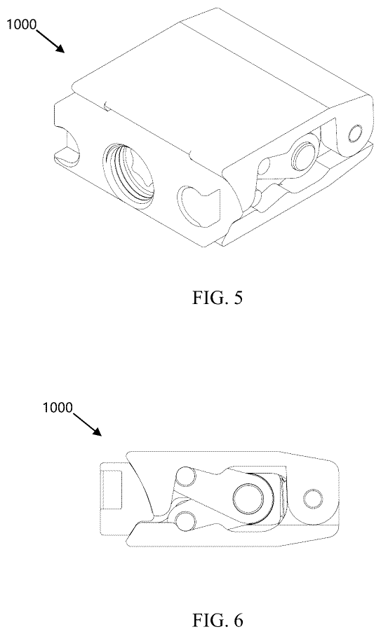

Referring now to FIGS. 1-7, one non-limiting expandable prosthetic device 1000 is illustrated. FIG. 1 illustrates the expandable prosthetic device 1000 in a fully expanded or open position and FIG. 5 illustrates the expandable prosthetic device 1000 is in the fully closed or collapsed position. The expandable prosthetic device 1000 includes a drive block 1100, a linkage block 1200, a drive screw 1300, a first endplate 1400, a second endplate 1500, and includes one or both of first and second sets of linkages 1600, 1610, wherein the first set of linkage 1600 includes linkages 1600 a, b, and the second set of linkage includes linkages 1610 a, b.

The drive block 1100 includes a first or proximal side 1102, a second or distal side 1104 opposite and spaced apart from the first side 1102, a third side 1106 extending between the first side 1102 and the second side 1104, a fourth side 1108 opposite and spaced apart from the third side 1106. The first end 1102 of the drive block 1100 defines a drive block opening 1120. The drive block 1100 includes a slot region 1160 that is located between the first side 1102, the second side 1104, the third side 1106 and the fourth side 1108 of the drive block 1100. The slot region 1160 can optionally include a top opening on the top side of the drive block 1100 and/or a bottom opening on the bottom side of the drive block 1100.

The drive block 1100 also includes first and second side slots 1170, 1180 that are positioned in the third side 1106 and fourth side 1108 respectively of the drive block 1100. The first side slot 1170 extends from the third side 1106 to the slot region 1160. The second side slot 1180 extends from the fourth side 1108 to the slot region 1160. The drive block opening 1120 extends from front end of the first or proximal side 1102 to the slot region 1160, and optionally extends along or parallel to the central axis of the drive block 1100.

The drive block opening 1120 has a first portion 1130 adjacent the front end of the first or proximal side 1102 and a second portion 1140 adjacent the first portion 1130. The first portion 1130 has a first diameter, and the second portion 1140 has a second diameter. The second diameter is optionally less than the first diameter; however, the diameters can be the same. The first portion 1130 optionally includes threading 1132. The second portion 1140 optionally is absent threading. In one non-limiting arrangement, the drive block opening 1120 is absent threading.

The optional threading in the first portion 1130, when threading is used, can be used to threadedly engage an insertion tool (not shown). The insertion tool can be releasable connected to the expandable prosthetic device 1000 via the threading in the first portion 1130. The insertion tool can be used to insert the expandable prosthetic device 1000 into a treatment area (e.g., between bones in foot, between bones in an ankle, between bones in the hand, between bones in the wrist, between bones in the spine, etc.). The insertion, when at least partially positioned in the drive block opening 1120 can optionally be configured to rotate the drive screw 1300 to cause the expandable prosthetic device 1000 to move between the fully closed or collapsed position and the fully open or expanded position, as will be discussed in more detail below. As illustrated in FIG. 7, the head 1310 of the drive screw 1300 can optionally include a screw opening 1320 that allows an insertion tool to releasably engage the head 1310 of the drive screw 1300 so as to facilitate in the rotation of the drive screw 1300 by the insertion tool. The shape of the screw opening 1320 is non-limiting (e.g., star-shaped, polygonal shaped, oval shaped, etc.).

The head 1310 of the drive screw 1300 can include a securing arrangement such as a slot 1330, rib, or some other arrangement that is used to facilitate in rotatable securing the head 1310 of the drive screw 1300 in the drive block opening 1120. In one non-limiting arrangement, a lock ring 1340 is at least partially inserted in a slot 1330 in the head 1310 of the drive screw 1300 to facilitate in rotatable securing the head 1310 of the drive screw 1300 in the drive block opening 1120. The drive block opening 1120 can include a block opening slot that also received a portion of the lock ring 1340 when the drive screw 1300 is rotatable secured in the drive block opening 1120.

When the head 1310 of the drive screw 1300 is rotatably positioned and secured in the drive block opening 1120, the end of the head 1310 of the drive screw 1300 can be configured such that the it does not extend outwardly from the drive block opening 1120. In one non-limiting arrangement, when the head 1310 of the drive screw 1300 is rotatably positioned and secured in the drive block opening 1120, the end of the head 1310 of the drive screw 1300 is recessed in the drive block opening 1120.

A bushing 1350 can optionally be used with the drive screw 1300 and be partially or fully positioned in the drive block opening 1120. The bushing 1350, when used, facilitates in the rotation of the head 1310 of the drive screw 1300 in the drive block opening 1120.

As illustrated in FIG. 7, the drive screw 1300 includes a body portion 1360 that includes threading. The body portion 1360 can have a diameter that is less than the head 1310 of the drive screw 1300. As will be discussed in more detail below, the threading on the body portion 1360 threadedly engages the linkage block 1200 and causes the linkage block 1200 to move relative to the drive block 1100 when the drive screw 1300 is rotated. When the expandable prosthetic device 1000 is fully assembled, all or a portion of the body portion 1360 extends into the slot region 1160 of the drive block 1100. The head 1310 of the drive screw 1300 can be configured such that the head 1310 cannot full pass through the drive block opening 1120.

The linkage block 1200 includes a linkage housing 1210 and a linkage bar 1230. The linkage housing 1210 includes a housing cavity 1212, a front opening 1214, a rear opening 1216, a first side opening 1218 and a second side opening 1220. The housing cavity 1212 opens to the front opening 1214, the rear opening 1216, the first side opening 1218 and the second side opening 1220. The linkage housing 1210 is configured to be positionable within the slot region 1160 of the drive block 1100 and be moveable within the slot region 1160 of the drive block 1100 along the longitudinal axis of the drive block 1100.

The linkage bar 1230 includes a linkable body 1232 and a first flange 1234 positioned at one end of the linkage body 1230 and a second flange 1236 positioned at the opposite end of the linkage body 1232. A linkage opening 1238 is located in the linkage body 1232. The linkage body 1230 is configured to be inserted through a first side opening 1218 and a second side opening 1220 of the linkage housing 1210 such that a) the first flange 1234 extends outwardly from the first side opening 1218 when the linkage bar 1230 is positioned in the linkage housing 1210, b) the second flange 1236 extends outwardly from the second side opening 1220 when the linkage bar 1230 is positioned in the linkage housing 1210, and c) the linkage opening 1238 is partially or fully aligned with the front opening 1214 and the rear opening 1216 when the linkage bar 1230 is positioned in the linkage housing 1210. The partial or full alignment of the linkage opening 1238 with the front opening 1214 and the rear opening 1216 when the linkage bar 1230 is positioned in the linkage housing 1210 allows a portion of the body portion 1360 of the drive screw 1300 to pass through the linkage opening 1238, the front opening 1214 and the rear opening 1216 when the expandable prosthetic device 1000 is fully assembled. The linkage opening 1238, the front opening 1214 and/or the rear opening 1216 can optionally include threading that is configured to engage threading on the body portion 1360 of the drive screw 1300. As illustrated in FIG. 7, the front opening 1214 and/or the rear opening 1216 include threading that is configured to engage threading on the body portion 1360 of the drive screw 1300, and the linkage opening 1238 is absent threading.

When the linkage bar 1230 is positioned in the linkage housing 1210, and the linkage housing 1210 is configured to be positionable within the slot region 1160 of the drive block 1100, a portion of the first flange 1234 is slidably positioned in the first side slot 1170 positioned in the third side 1106 of the drive block 1100, and a portion of the second flange 1236 is slidably positioned in the second side slot 1180 positioned in the fourth side 1108 of the drive block 1100. When the drive screw 1300 is rotated, the linkage housing 1210 moves within the slot region 1160 of the drive block 1100 and the sliding movement of the portion of the first flange 1234 in the first side slot 1170 and the portion of the second flange 1236 in the second side slot 1180 facilitates in the guiding of the linkage housing 1210 as it moves in the slot region 1160 of the drive block 1100.

In one non-limiting arrangement, the size and configuration of the linkage housing 1210 is such that it is fully contained within the slot region 1160 of the drive block 1100 when the expandable prosthetic device 1000 is fully assembled. In another non-limiting arrangement, the size and configuration of the linkage housing 1210 is such that it is fully contained within the slot region 1160 of the drive block 1100 when the expandable prosthetic device 1000 is fully assembled and when the linkage housing 1210 moves within the slot region 1160 of the drive block 1100. In another non-limiting arrangement, the size and configuration of the linkage housing 1210 is such that a portion of the top and/or bottom of the linkage housing 1210 is positioned within at least a portion of the top opening 1162 and/or the bottom opening 1164 of the slot region 1160 of the drive block 1100 when the expandable prosthetic device 1000 is fully assembled an when the linkage housing 1210 moves within the slot region 1160 of the drive block 1100 so as to facilitate in the guiding of the linkage housing 1210 as it moves in the slot region 1160 of the drive block 1100. In another non-limiting arrangement, the ends of the end of the first flange 1234 does not extend outwardly from the first side slot 1170 and/or the end of the second flange 1236 does not extend outwardly from the second side slot 1180 as the linkage housing 1210 moves in the slot region 1160 of the drive block 1100.

Each of the first endplate 1400 and the second endplate 1500 include a body 1410, 1510. The body 1410, 1510 has a top side 1412, 1512 and a bottom side opposite and spaced apart from the first side 1412, 1512.

The first sides 1412, 1512 of the first and second endplates 1400, 1500 can optionally include a graft window (not shown). The graft windows can optionally be used to facilitate bone growth once the expandable prosthetic device is inserted in a patient.

The first sides 1412, 1512 of the first and/or second endplates 1400, 1500 can optionally include a micro-textured surface and/or one or more teeth to create friction between the bone and the endplates. However, in other implementations, the first sides 1412, 1512 of the first and/or second endplates 1400, 1500 can include any number of teeth, no teeth, and/or no micro-textured surface.

The first and second endplates 1400, 1500 can include planar top surfaces that do not lie within the same plane. As illustrated in FIGS. 1-2, the first and second endplates 1400, 1500 both include a front portion 1414, 1514 and a rear portion 1416, 1516 when the top surface of these two portions do not lie in the same plane. The rear portion 1416 of the first endplate 1400 slopes downwardly from the front portion 1414 at an angle of about 5-60° (and all values and ranges therebetween). The top surface of the front portion 1414 of the first endplate 1400 has a generally planar or flat surface along 50-100% (and all values and ranges therebetween) of the longitudinal length of the front portion 1414. The longitudinal length of the rear portion 1416 is generally less than the longitudinal length of the front portion 1414. Generally, the longitudinal length of the rear portion 1416 is 20-80% (and all values and ranges therebetween) of the longitudinal length of the front portion 1414. As also illustrated in FIGS. 1-2, the rear portion 1516 of the second endplate 1500 slopes downwardly from the front portion 1514 at an angle of about 5-60° (and all values and ranges therebetween). The top surface of the front portion 1514 of the second endplate 1500 has a generally planar or flat surface along 50-100% (and all values and ranges therebetween) of the longitudinal length of the front portion 1514. The longitudinal length of the rear portion 1516 is generally less than the longitudinal length of the front portion 1514. Generally, the longitudinal length of the rear portion 1516 is 20-80% (and all values and ranges therebetween) of the longitudinal length of the front portion 1514.

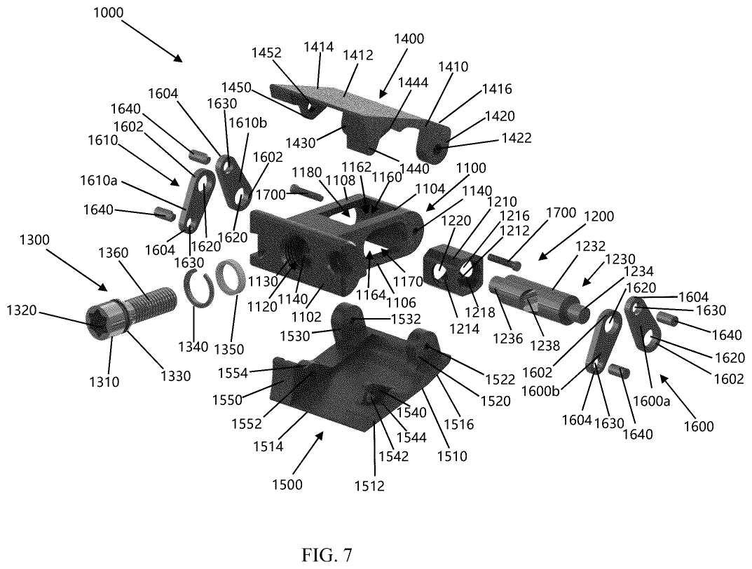

As illustrated in FIG. 6, the plane of the top surface of the front portion 1414 of the first endplate 1400 is generally parallel (e.g., +5° and all values and ranges therebetween) to the plane of the of the top surface of the front portion 1514 of the second endplate 1500 when the expandable prosthetic device 1000 is in the fully closed or collapsed position. As illustrated in FIG. 6, the front ends of the first and second endplates 1400, 1500 can be configured to not contact one another as the expandable prosthetic device 1000 moves from the fully closed or collapsed position to the fully open or expanded position.

The expandable prosthetic device 1000 includes first and second sets of linkages 1600, 1610, wherein the first set of linkage 1600 includes linkages 1600 a, b, and the second set of linkage includes linkages 1610 a, b. Each of the four linkages 1600a, 1600b, 1600c, 1600d has a longitudinal axis, a first portion 1602, and a second portion 1604 spaced apart from the first portion 1602 along the longitudinal axis.

The first portions 1602 of linkages 1600a, 1600b are rotatably coupled to the first flange 1234 of the linkage bar 1230 of the linkage block 1200, and the first portions 1602 of linkages 1610a, 1610b are rotatably coupled to the second flange 1236 of the linkage bar 1230 of the linkage block 1200. The first portions of each of linkages 1600a, 1600b, 1610a, 1610b includes a flange opening 1620 that is configured to receive a portion of first or second flanges 1234, 1236 of linkage bar 1230. In one non-limiting arrangement, as illustrated in FIG. 1, the first and second linkages 1600a, 1600b rotatably engage and/or are rotatably coupled to the first flange 1234 of the linkage bar 1230 of the linkage block 1200 along the same rotation axis, and the third and fourth linkages 1610a, 1610b rotatably engage and/or are rotatably coupled to the second flange 1236 of the linkage bar 1230 of the linkage block 1200 along the same rotation axis. In one non-limiting embodiment, the same rotation axis is shared by all four linkages when rotatably engage and/or are rotatably coupled to the linkage bar 1230 of the linkage block 1200. In another non-limiting embodiment, the same rotation axis is shared by all four linkages when connected to the linkage bar 1230 of the linkage block 1200, and the rotation axis is along the central longitudinal axis of the linkage bar 1230. As illustrated in FIG. 1, at least a portion of the first and second linkages 1600a, 1600b overlie one another along the rotation axis of the linkages, and at least a portion of the third and fourth linkages 1610a, 1610b overlie one another along the rotation axis of the linkages. As illustrated in FIG. 1, linkage 1600b is located closer to linkage opening 1238 of linkage body 1232 than linkage 1600a. Likewise, linkage 1610b is located closer to linkage opening 1238 of linkage body 1232 than linkage 1610a.

The second portions 1604 of linkages 1600a, 1610b are configured to engage, rotatably engage and/or rotatably coupled to the first endplate 1400, and the second portions 1604 of linkages 1600b, 1610a are configured to engage, rotatably engage and/or rotatably coupled to the second endplate 1500.

The second portions 1604 of linkages 1600a, 1610b and the second portions 1604 of linkages 1600b, 1610a include a pin opening 1630 that is configured to receive a pin 1640. The pin 1640 can be rigidly connected in the pin opening 1630 of one or more or all of linkages 1600a, 1610b, 1600b, 1610a, rotatably engage the pin opening 1630 of one or more or all of linkages 1600a, 1610b, 1600b, 1610a, or be rotatably connected to the pin opening 1630 of one or more or all of linkages 1600a, 1610b, 1600b, 1610a. In one non-limiting arrangement, pin 1640 is rigidly connected in the pin opening 1630 of each linkages 1600a, 1610b, 1600b, 1610a. In another non-limiting arrangement, pin 1640 rotatably engages the pin opening 1630 of each linkages 1600a, 1610b, 1600b, 1610a, and may or may not be rotatably connected to the pin opening 1630 of each linkages 1600a, 1610b, 1600b, 1610a.

Referring again to FIGS. 1 and 7, the first endplate 1400 includes first and second rear pivot flanges 1420, 1430 positioned on opposite sides of the first endplate 1400 and extend downwardly from the rear portion 1416 of the body 1410 of the first endplate 1400. Each of the rear pivot flanges 1420, 1430 includes a pivot opening 1422, 1432. Rear pivot flange 1420 is positioned closer to the side edge of the body 1410 of the first endplate 1400 than rear pivot flange 1430; however, this is not required. The front portion 1414 of the body 1410 of the first endplate 1400 includes front flanges 1440, 1450 and are position on opposite sides of the first endplate 1400 and extend downwardly from the front portion 1414 of the body 1410 of the first endplate 1400. Each of the front flanges 1440, 1450 includes an opening 1442, 1452. Front flange 1440 has a thicker profile than front pivot flange 1450. Also, front flange 1440 extends farther downwardly from front portion 1414 of the body 1410 as compared to front flange 1450; however, this is not required.

Each of the front flanges 1440, 1450 includes a linkage landing 1444, 1454 that has a generally V-shaped profile. The linkage landing 1444 is configured to be engaged by the end edge of the second portion 1604 of linkage 1600a when a portion of pin 1640 is positioned in opening 1442 of front flange 1440. The linkage landing 1454 is configured to be engaged by the end edge of the second portion 1604 of linkage 1610b when a portion of pin 1640 is positioned in opening 1452 of front flange 1450. The portion of pin 1640 that is positioned in openings 1442 and 1452 can be a) rotatably engaged in openings 1442 and 1452, and/or b) rotatably connected to openings 1442 and 1452. In one non-limiting arrangement, the engagement between the end edge of the second portion 1604 of linkage 1600a and the linkage landing 1444, and the engagement between the end edge of the second portion 1604 of linkage 1610b and the linkage landing 1454 provide 60-100% (and all values and ranges therebetween) of the movement force to the underside of the first endplate 1400 when the expandable prosthetic device 1000 moves from the fully close or collapse position toward the fully open or expanded position, and the portion of pin 1640 that is positioned in openings 1442 and 1452 a) only rotatably engages with openings 1442 and 1452 and is rigidly connected to the pin opening 1630 of linkages 1600a, 1610b, and applies 0-100% (e.g., 0-100% and all values and ranges therebetween) of the movement force to the underside of the first endplate 1400 when the expandable prosthetic device 1000 moves from the fully close or collapse position toward the fully open or expanded position, or b) is rigidly connected in openings 1442 and 1452 and is rotatably connected to the pin opening 1630 of linkages 1600a, 1610b, and applies 0-100% (e.g., 0-100% and all values and ranges therebetween) of the movement force to the underside of the first endplate 1400 when the expandable prosthetic device 1000 moves from the fully close or collapse position toward the fully open or expanded position.

As illustrated in FIG. 7, the bottom profile and structures of the second endplate 1500 are a mirror image of the bottom profile and structures of the first endplate 1400; however, this is not required. The second endplate 1500 includes first and second rear pivot flanges 1520, 1530 positioned on opposite sides of the second endplate 1500 and extend upwardly from the rear portion 1516 of the body 1510 of the second endplate 1500. Each of the rear pivot flanges 1520, 1530 includes a pivot opening 1522, 1532. Rear pivot flange 1520 is positioned farther to the side edge of the body 1510 of the second endplate 1500 than rear pivot flange 1530; however, this is not required. The front portion 1514 of the body 1510 of the second endplate 1500 includes front flanges 1540, 1550 and are position on opposite sides of the first endplate 1500 and extend upwardly from the front portion 1514 of the body 1510 of the second endplate 1500. Each of the front flanges 1540, 1550 includes an opening 1542, 1552. Front flange 1550 has a thicker profile than front flange 1540. Also, front flange 1550 extends farther upwardly from front portion 1514 of the body 1510 as compared to front flange 1540; however, this is not required.

Each of the front flanges 1540, 1550 includes a linkage landing 1544, 1554 that has a generally V-shaped profile. The linkage landing 1544 is configured to be engaged by the end edge of the second portion 1604 of linkage 1600b when a portion of pin 1640 is positioned in opening 1542 of front flange 1540. The linkage landing 1554 is configured to be engaged by the end edge of the second portion 1604 of linkage 1610a when a portion of pin 1640 is positioned in opening 1552 of front flange 1550. The portion of pin 1640 that is positioned in openings 1542 and 1552 can be a) rotatably engaged in openings 1542 and 1552, and/or b) rotatably connected to openings 1542 and 1552. In one non-limiting arrangement, the engagement between the end edge of the second portion 1604 of linkage 1600b and the linkage landing 1544, and the engagement between the end edge of the second portion 1604 of linkage 1610a and the linkage landing 1554 provide 60-100% (and all values and ranges therebetween) of the movement force to the underside of the second endplate 1500 when the expandable prosthetic device 1000 moves from the fully close or collapse position toward the fully open or expanded position, and the portion of pin 1640 that is positioned in openings 1542 and 1552 a) only rotatably engages with openings 1542 and 1552 and is rigidly connected to the pin opening 1630 of linkages 1600b, 1610a, and applies 0-100% (e.g., 0-100% and all values and ranges therebetween) of the movement force to the underside of the second endplate 1500 when the expandable prosthetic device 1000 moves from the fully close or collapse position toward the fully open or expanded position, or b) is rigidly connected in openings 1542 and 1552 and is rotatably connected to the pin opening 1630 of linkages 1600b, 1610a, and applies 0-100% (e.g., 0-100% and all values and ranges therebetween) of the movement force to the underside of the second endplate 1500 when the expandable prosthetic device 1000 moves from the fully close or collapse position toward the fully open or expanded position.

In one non-limiting arrangement, the portion of pin 1640 that is positioned in openings 1442 and 1452 is only rotatably engaged with openings 1442 and 1452 and is rigidly connected to the pin opening 1630 of linkages 1600a, 1610b, and applies no more than 50% (e.g., 0-50% and all values and ranges therebetween) of the movement force to the underside of the first endplate 1400 when the expandable prosthetic device 1000 moves from the fully close or collapse position toward the fully open or expanded position, and a portion of pin 1640 that is positioned in openings 1542 and 1552 is only rotatably engaged with openings 1542 and 1552 and is rigidly connected to the pin opening 1630 of linkages 1600b, 1610a, and applies no more than 50% (e.g., 0-50% and all values and ranges therebetween) of the movement force to the underside of the second endplate 1500 when the expandable prosthetic device 1000 moves from the fully close or collapse position toward the fully open or expanded position.

Referring again to FIG. 7. Each side of the rear portion of the drive block 1110 includes openings 1140 that are configured to receive pivot pin 1700. One pivot pin 1700 is configured to be inserted in pivot opening 1422 of rear pivot flange 1420 of first endplate 1400, and also inserted in pivot opening 1522 of rear pivot flange 1520 of second endplate 1500, and also into opening 1140 on one side of the rear portion of the drive block 1110, and the other pivot pin 1700 is configured to be inserted in pivot opening 1432 of rear pivot flange 1430 of first endplate 1400, and also inserted in pivot opening 1532 of rear pivot flange 1530 of second endplate 1500, and also into opening 1140 on the other side of the rear portion of the drive block 1110. The openings 1140 can optionally include threading to threadedly receive a threaded portion of the pivot pin 1700; however, this is not required. Generally, pivot pin 1700 rotatably engages pivot openings 1422, 1432, 1522, 1532 and is generally not threadedly engaged to such openings. The pivot connection formed by pivot pins 1700 results in the rear portion 1416 of the body 1410 of the first endplate 1400 and the rear portion 1516 of the body 1510 of the second endplate 1500 being pivotally connected to rear portion of the drive block 1110. In one non-limiting arrangement, the pivot axis of pivot openings 1422, 1432, 1522, 1532 and openings 1140 are the same. The pivot connection formed by pivot pins 1700 results in the end portion of rear portion 1416 of the body 1410 of the first endplate 1400 and the end portion of the rear portion 1516 of the body 1510 of the second endplate 1500 being maintained at a generally constant distance from one another as the expandable prosthetic device 1000 moves from the fully close or collapse position toward the fully open or expanded position.

The expandable prosthetic device 1000 when being used, the first and second endplates 1400, 1500 of the expandable prosthetic device 1000 begin at a minimum distance from each other (a minimum “height”) in a fully closed or collapsed position, as illustrated in FIG. 5-6. A rotation tool or insertion tool (not illustrated) is inserted into the drive block opening 1120 until the rotation tool or insertion tool engages screw opening 1320 of the head 1310 of the drive screw 1300. The drive screw 1300 is rotated within the drive block opening 1120 and causes the linkage block 1200 to move within the slot region 1160 of the drive block 1100. When the linkage block 1200 is caused to move within the slot region 1160 of the drive block 1100 and toward the drive block opening 1120, the front portions 1414, 1514 of the first and second endplates 1400, 1500 are cause to separate form one another.

During rotation of the drive screw 1300, head 1310 of the drive screw 1300 does not move axially along the longitudinal axis of the drive block while the linkable block 1200 moves relative to the drive block 1100. In one non-limiting arrangement, a portion of the body portion 1360 of the drive screw 1300 extends outwardly from a second end of the drive block opening 1120, and into slot region 1160 of the drive block 1100, and into the first side opening 1218 and a second side opening 1220 of the linkage housing 1210 and the linkage opening 1238 of linkage body 1232. During rotation of the drive screw 1300, the threaded portion of body portion 1360 of the drive screw 1300 engages threading on one or more of the first side opening 1218 and/or the second side opening 1220 of the linkage housing 1210, and/or the linkage opening 1238 of linkage body 1232 to cause the linkage block 1200 to move in the slot region 1160 of the drive block 1100. As the linkage block 1200 moves in the slot region 1160 of the drive block 1100 and toward the second end of the drive block opening 1120, the expandable prosthetic device 1000 is cause to move toward the fully open or expanded position as illustrated in FIGS. 1-4.

As the linkage block 1200 moves from the fully closed or collapsed position of the expandable prosthetic device 1000 to the fully open or expanded position, the rotational axis of the first portions 1600a, 1600b, 1610a, 1610b of the linkages 1600a, 1600b, 1610a, 1610b moves toward the second end of the drive block opening 1120 of the drive block 1100, the relative angles of the longitudinal axes of each of the linkages 1600a, 1600b, 1610a, 1610b increase with respect to the central axis of the linkage block 1200. Because the second ends of linkages 1600a, 1610b rotatably engage the first endplate 1400 and the second ends of the linkages 1600b, 1610s rotatably engage the second endplate 1500, the increasing relative angles of the longitudinal axes of the linkages 1600a, 1600b, 1610a, 1610b with respect to the central axis of the linkage block 1200 causes the first and second endplates 1400, 1500 to move away from each other until the expandable prosthetic device 1000 reaches the fully open or expanded position, as illustrated in FIGS. 1-4. Thus, rotation of the drive screw 1300 can simultaneously cause movement of the linkage block 1200 relative to the drive block 1100 and movement of the first endplate 1400 relative to the second endplate 1500.

As the front portions 1414, 1514 of the first and second endplates 1400, 1500 move away from each other and away from one another, the rear portions 1416, 1516 of the first and second endplates 1400, 1500 pivot about the rear end portion of the drive block 1100 about the axis of pivot pin 1700.

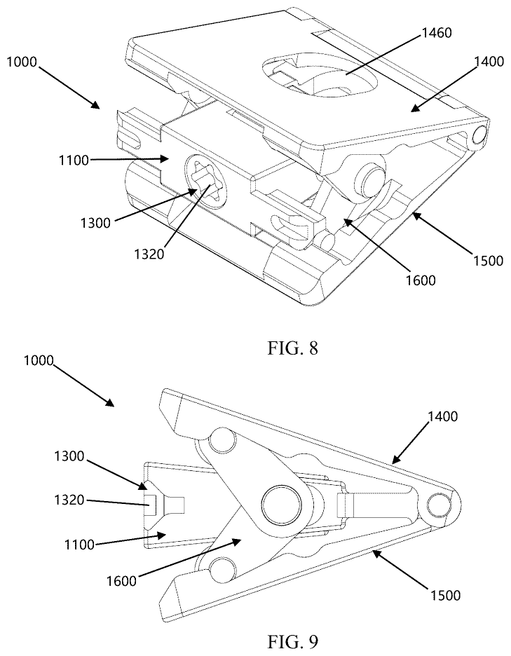

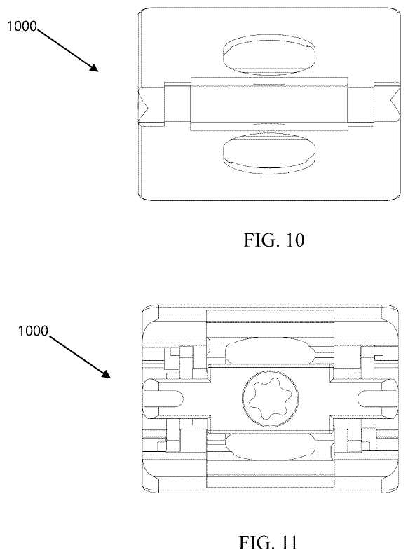

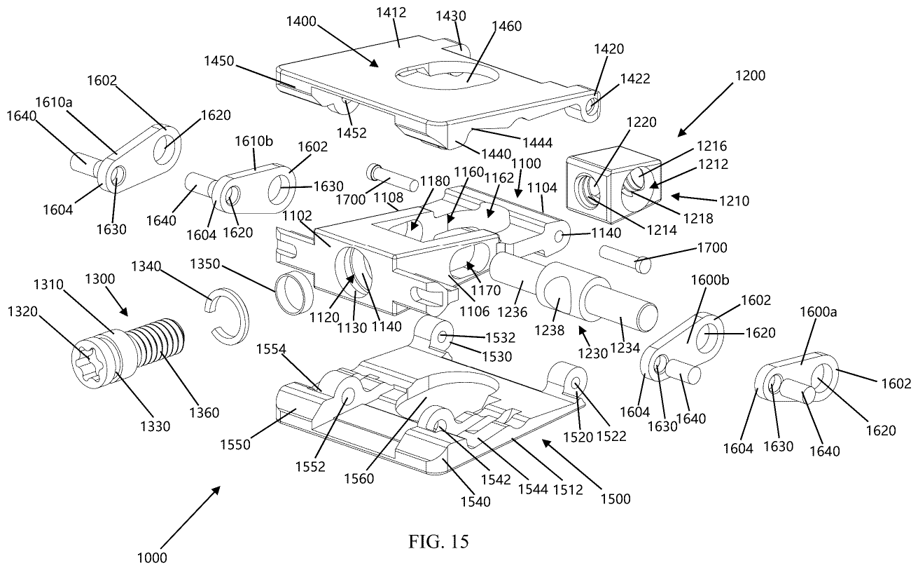

Referring now to FIGS. 8-15, another non-limiting embodiment of the expandable prosthetic device 1000 is illustrated. The expandable prosthetic device 1000 illustrated in FIGS. 8-15 is configured similarly to the expandable prosthetic device 1000 illustrated in FIGS. 1-7 and also operates similarly to the expandable prosthetic device 1000 illustrated in FIGS. 1-7. As such, similar components of the expandable prosthetic device 1000 illustrated in FIGS. 1-15 will be numbered the same, and the operation for expandable prosthetic device 1000 illustrated in FIGS. 8-15 and the function of the components of the expandable prosthetic device 1000 illustrated in FIGS. 8-15 are the same as the expandable prosthetic device 1000 illustrated in FIGS. 1-7 unless indicated otherwise below.

As illustrated in FIGS. 8, 12, 13 and 15, the first sides 1412, 1512 of the first and second endplates 1400, 1500 include a graft window 1460, 1560. The graft windows can optionally be used to facilitate bone growth once the expandable prosthetic device is inserted in a patient.

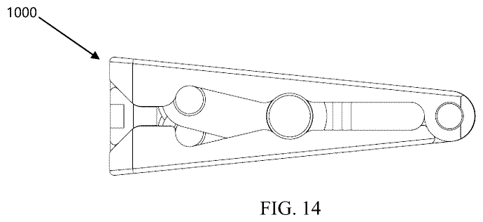

Unlike the expandable prosthetic device 1000 as illustrated in FIG. 6, the plane of the top surface of the front portion 1414 of the first endplate 1400 when the expandable prosthetic device 1000 is the fully closed or collapsed position is not parallel to the plane of the of the top surface of the front portion 1514 of the second endplate 1500 as illustrated in FIG. 14. The angle of the planes of the top surface of the front portion 1414 of the first endplate 1400 to the plane of the of the top surface of the front portion 1514 of the second endplate 1500 when the expandable prosthetic device 1000 is the fully closed or collapsed position is about 5-30° (and all values and ranges therebetween). As illustrated in FIG. 14, the front ends of the first and second endplates 1400, 1500 can be configured to not contact one another as the expandable prosthetic device 1000 moves from the fully closed or collapsed position to the fully open or expanded position.

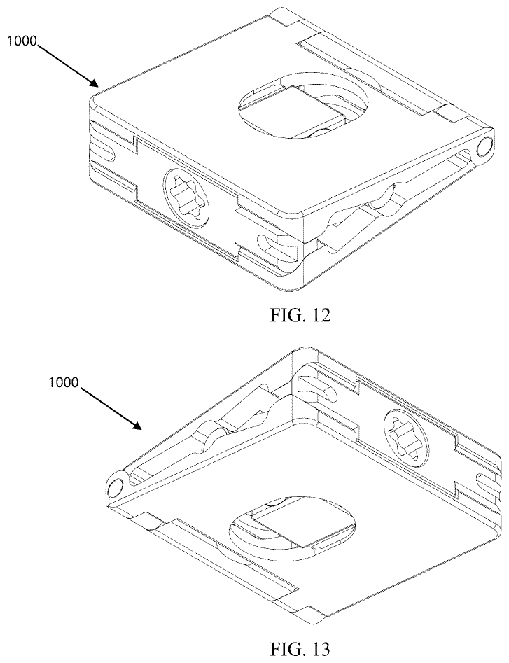

The expandable prosthetic device 1000 of FIGS. 8-15, when being used, operates very similar to the operation of the expandable prosthetic device 1000 of FIGS. 1-7. The first and second endplates 1400, 1500 of the expandable prosthetic device 1000 begin at a minimum distance from each other (a minimum “height”) in a fully closed or collapsed position, as illustrated in FIG. 12-14. A rotation tool or insertion tool (not illustrated) is inserted into the drive block opening 1120 until the rotation tool or insertion tool engages screw opening 1320 of the head 1310 of the drive screw 1300. The drive screw 1300 is rotated within the drive block opening 1120 and causes the linkage block 1200 to move within the slot region 1160 of the drive block 1100. When the linkage block 1200 is caused to move within the slot region 1160 of the drive block 1100 and toward the drive block opening 1120, the front portions 1414, 1514 of the first and second endplates 1400, 1500 are cause to separate form one another.

During rotation of the drive screw 1300, head 1310 of the drive screw 1300 does not move axially along the longitudinal axis of the drive block while the linkable block 1200 moves relative to the drive block 1100. In one non-limiting arrangement, a portion of the body portion 1360 of the drive screw 1300 extends outwardly from a second end of the drive block opening 1120, and into slot region 1160 of the drive block 1100, and into the first side opening 1218 and a second side opening 1220 of the linkage housing 1210 and the linkage opening 1238 of linkage body 1232. During rotation of the drive screw 1300, the threaded portion of body portion 1360 of the drive screw 1300 engages threading on one or more of the first side opening 1218 and/or the second side opening 1220 of the linkage housing 1210, and/or the linkage opening 1238 of linkage body 1232 to cause the linkage block 1200 to move in the slot region 1160 of the drive block 1100. As the linkage block 1200 moves in the slot region 1160 of the drive block 1100 and toward the second end of the drive block opening 1120, the expandable prosthetic device 1000 is cause to move toward the fully open or expanded position as illustrated in FIGS. 8-11.

As the linkage block 1200 moves from the fully closed or collapsed position of the expandable prosthetic device 1000 to the fully open or expanded position, the rotational axis of the first portions 1600a, 1600b, 1610a, 1610b of the linkages 1600a, 1600b, 1610a, 1610b moves toward the second end of the drive block opening 1120 of the drive block 1100, the relative angles of the longitudinal axes of each of the linkages 1600a, 1600b, 1610a, 1610b increase with respect to the central axis of the linkage block 1200. Because the second ends of linkages 1600a, 1610b rotatably engage the first endplate 1400 and the second ends of the linkages 1600b, 1610s rotatably engage the second endplate 1500, the increasing relative angles of the longitudinal axes of the linkages 1600a, 1600b, 1610a, 1610b with respect to the central axis of the linkage block 1200 causes the first and second endplates 1400, 1500 to move away from each other until the expandable prosthetic device 1000 reaches the fully open or expanded position, as illustrated in FIGS. 8-11. Thus, rotation of the drive screw 1300 can simultaneously cause movement of the linkage block 1200 relative to the drive block 1100 and movement of the first endplate 1400 relative to the second endplate 1500.

As the front portions 1414, 1514 of the first and second endplates 1400, 1500 move away from each other and away from one another, the rear portions 1416, 1516 of the first and second endplates 1400, 1500 pivot about the rear end portion of the drive block 1100 about the axis of pivot pin 1700.

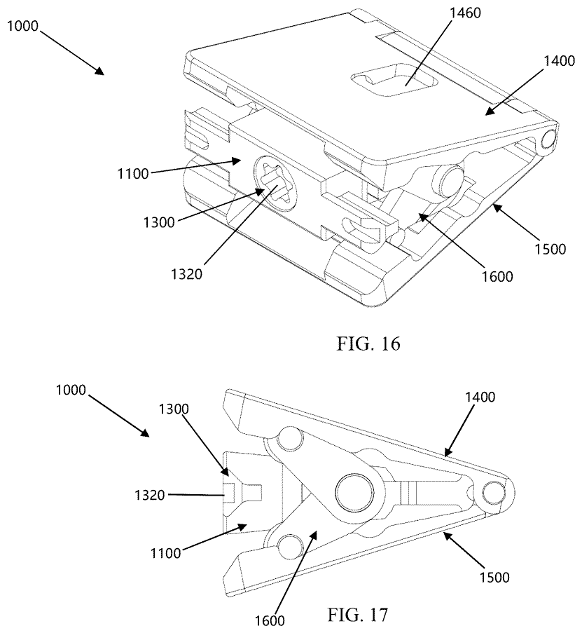

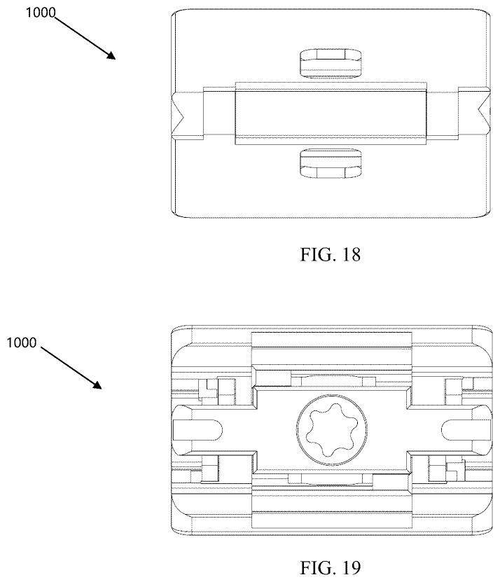

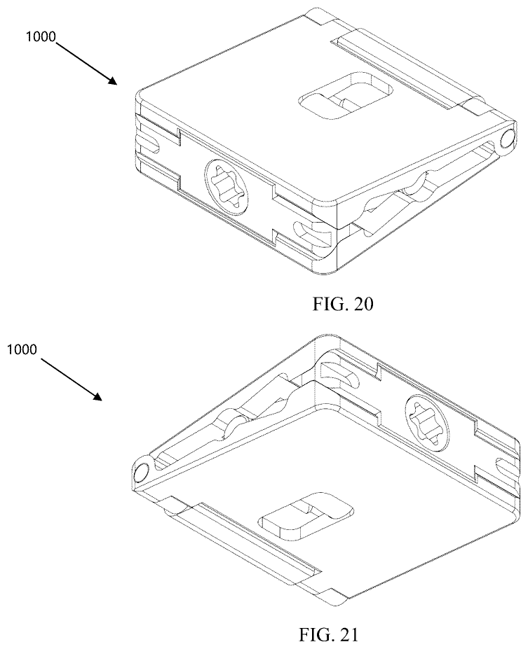

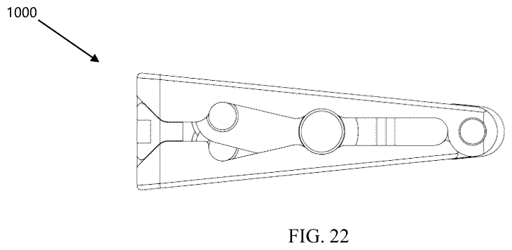

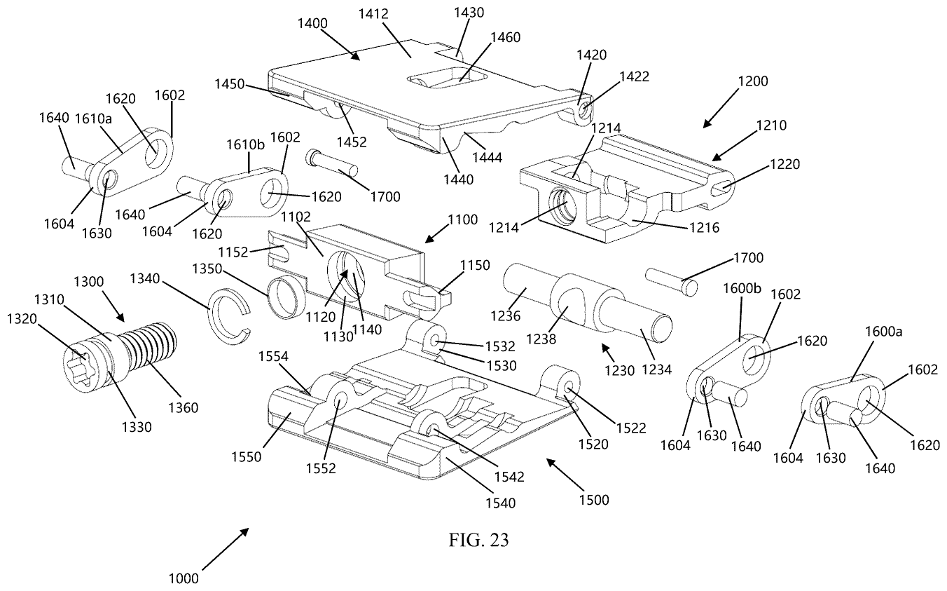

Referring now to FIGS. 16-23, another non-limiting embodiment of the expandable prosthetic device 1000 is illustrated. The expandable prosthetic device 1000 illustrated in FIGS. 16-23 is configured similarly to the expandable prosthetic device 1000 illustrated in FIGS. 8-15 and also operates similarly to the expandable prosthetic device 1000 illustrated in FIGS. 8-15. However, the drive block 1100 and linkage block 1200 are configured differently from the drive block 1100 and linkage block 1200 of the expandable prosthetic device 1000 that is illustrated in FIGS. 1-15, and the operation of the drive block 1100 and linkage block 1200 of the prosthetic device 1000 illustrated in FIGS. 16-23 operates differently from the drive block 1100 and linkage block 1200 of the expandable prosthetic device 1000 that is illustrated in FIGS. 1-15 as will be discussed below.

The drive block 1100 includes drive block opening 1120 that has a first portion 1130 adjacent the front end of the first side 1102 and a second portion 1140 adjacent the first portion 1130. The drive block opening 1120 is typically the only opening in the drive block along the longitudinal length of the drive block 1100. The drive block 1100 has two side flanges 1150, 1152 that are positioned on the front side portions of the drive block 1100 and extend outwardly at an angle that is generally transverse to the longitudinal axis of the drive block 1100. Such side flanges can also be used in the drive blocks 1100 illustrated in FIGS. 1-15. Unlike the drive blocks 1100 illustrated in FIGS. 1-15, drive block 1100 illustrated in FIGS. 16-23 is absent structures rearwardly of the drive block opening 1120. Also, unlike the drive blocks 1100 illustrated in FIGS. 1-15, drive block 1100 is not connected to rear end portions of the first endplate 1400 and second endplate 1500. The read side of the drive block opening 1120 is positioned forwardly of linkage block 1200 and little or no portion of linkage block 1200 is contained in and/or moveable within drive block 1100. The drive block 1100 is optionally no connected to one or both of the first endplate 1400 and second endplate 1500. In such arrangement, the drive block 1100 can optionally be only connected to linkage block 1200 via drive screw 1300.

The linkage housing 1210 of the linkage block 1200 includes a rear portion that includes pin slots 1220 that are configured to receive a portion of pivot pin 1700. The pin slots 1220 are enlongated to enable the linkage housing to move along a longitudinal axis of the expandable prosthetic device 1000 and relative to the fixed longitudinal position of the rear ends of the first endplate 1400 and second endplate 1500 when the drive screw 1300 is rotated. The longitudinal movement of the linkage block 1200 results in the rotation of the linkages 1600a, 1600b, 1610a, 1610b about the longitudinal axis (e.g., central longitudinal axis) of the linkage bar 1230, thereby resulting in the movement of the proximal end portions of the first endplate 1400 and second endplate 1500 relative to one another as the distal and portions of the first endplate 1400 and second endplate 1500 pivot about pivot pin 1700.

In the arrangement of the expandable prosthetic device 1000 illustrated in FIGS. 16-23, when the proximal end portions of the first endplate 1400 and second endplate 1500 move relative to one another between the fully closed or collapsed position and the fully open or expanded position, the front end or proximal end of the drive block 1100 remains generally in the same plane as the proximal ends of the first endplate 1400 and second endplate 1500 as illustrated in FIGS. 16-22. The drive block 1100 is optionally able to move a limited distance toward the linkage block 1200 and/or the rear ends of the first endplate 1400 and second endplate 1500 when the drive screw 1300 is rotated and as the first endplate 1400 and second endplate 1500 move from the fully closed or collapsed position to the fully open or expanded position. For the arrangement of the expandable prosthetic device 1000 illustrated in FIGS. 1-15, the drive block 1100 does not move toward rear ends of the first endplate 1400 and second endplate 1500 when the drive screw 1300 is rotated and as the first endplate 1400 and second endplate 1500 move from the fully closed or collapsed position to the fully open or expanded position, thereby resulting in the proximal ends of the first endplate 1400 and second endplate 1500 moving rearwardly of the proximal end of the drive block 1100 as the first endplate 1400 and second endplate 1500 move from the fully closed or collapsed position to the fully open or expanded position as illustrated in FIGS. 1-6 and 8-14. The optionally movement of the drive block 1100 toward rear ends of the first endplate 1400 and second endplate 1500 when the drive screw 1300 is rotated can facilitate is wedging apart the front end regions of the first endplate 1400 and second endplate 1500 when the drive screw 1300 is rotated. Such wedging opening effect can be achieve by the contact of sloped flange surface 1150 with sloped surfaces 1450, 1150 on the first endplate 1400 and second endplate 1500.

As the drive screw 1300 is rotated, the linkage block 1200 is caused to immediately or eventually move longitudinally forward toward the front ends of the first endplate 1400 and second endplate 1500. As the linkage block 1200 moves longitudinally forwardly, the linkage bar 1230 that is positioned in the linkage block 1200 is also caused to move longitudinally forwardly. The linkage bar 1230 is configured to be maintained in position relative to the linkage block 1200 as the linkage block 1200 moves longitudinally forwardly. This is a different configuration from expandable prosthetic device 1000 that is illustrated in FIGS. 1-15 wherein the linkage block 1200 as the linkage block 1200 are configured to move longitudinally forwardly within drive block 1100.

One or more or all of the components of the expandable prosthetic device 1000 can be partially or fully formed of a metal alloy. In one non-limiting embodiment, a portion or all of the one or more or all of the components of the expandable prosthetic device is formed of a metal alloy selected from a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) refractory metal alloy, or l) metal alloy that includes at least 5 awt. % rhenium. The material used to form the different components of the expandable prosthetic device 1000 can be the same or different.

One or more portions of the outer surface of the expandable prosthetic device 1000 can be coated with an enhancement layer. Non-limiting enhancement layers include chromium nitride (CrN), diamond-like carbon (DLC), titanium nitride (TiN), titanium oxynitride or titanium nitride oxide (TiNOₓ), zirconium nitride (ZrN), zirconium oxide (ZrO₂), zirconium oxynitride (ZrNₓO_{y}) [e.g., cubic ZrN:O, cubic ZrO₂:N, tetragonal ZrO₂:N, and monoclinic ZrO₂:N phase coatings], oxyzirconium-nitrogen-carbon (ZrNC), zirconium OxyCarbide (ZrOC), and combinations of such coatings. The one or more enhancement layers, when used, can be optionally applied to a portion or all of the outer surface of the expandable prosthetic device 1000 by use of a physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process. The thickness of the enhancement layer, when used, is greater than 1 nanometer.

The expandable prosthetic device 1000 can optionally be partially or fully be coated with and/or include one or more agents.

One or more portions of the expandable prosthetic device 1000 can optionally include a marker material that facilitates enabling the expandable prosthetic device 1000.

A number of implementations have been described. Nevertheless, it will be understood that various modifications may be made without departing from the spirit and scope of the claims. Accordingly, other implementations are within the scope of the following claims.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall therebetween.

To aid the Patent Office and any readers of this application and any resulting patent in interpreting the claims appended hereto, applicants do not intend any of the appended claims or claim elements to invoke 35 U.S.C. 112 (f) unless the words “means for” or “step for” are explicitly used in the particular claim.

What is claimed:

1. An expandable prosthetic device; said expandable prosthetic device comprises:
a drive block; said drive block includes a drive block opening and a slot region;
a linkage block; said linkage block includes a linkage housing and a linkage bar; said linkage bar is at least partially positioned in said linkage housing; said linkage block is movable within said slot region of said drive block;
a drive screw; said drive screw includes a head and body portion; said head is rotatably positioned in within said drive block opening; said body portion is threadingly connected to said linkage block;
first and second endplates; said first endplate includes a front portion and a rear portion; said second endplate includes a front portion and a rear portion; said rear portion of said first and second endplates are pivotally connected together; and,
first and second sets of linkages; said first set of linkage includes first and second linkages; said second set of linkage includes first and second linkages; said first linkage of said first set of linkages engages said linkage bar and said first endplate; said first linkage of said second set of linkages engages said linkage bar and said first endplate; said second linkage of said first set of linkages engages said linkage bar and said second endplate; said second linkage of said second set of linkages engages said linkage bar and said second endplate; said first and second sets of linkages are spaced from one another; and
wherein rotation of said drive screw simultaneously causes I) movement of said linkage block relative to said drive block, and II) movement of said first endplate relative to said second endplate.

2. The expandable prosthetic device as defined in claim 1, wherein said drive screw is rotatably coupled within said drive block opening and is threadingly disposed within said linkage block.

3. The expandable prosthetic device as defined in claim 1, wherein each linkage has a first portion and a second portion that is spaced apart from said first portion along a longitudinal axis; said first portion of each of said linkages is rotatably coupled to said linkage bar and said second portion of each of said linkages engages either said first or second endplate.

4. The expandable prosthetic device as defined in claim 1, wherein at least a portion of said expandable prosthetic device includes one or more metal alloys selected from the group consisting of a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) refractory metal alloy, or l) metal alloy that includes at least 5 awt. % rhenium.

5. The expandable prosthetic device as defined in claim 1, wherein one or more of said first endplate and said second endplate includes a micro-textured surface, and/or one or more teeth.

6. The expandable prosthetic device as defined in claim 1, wherein one or more portions of said expandable prosthetic device includes an enhancement layer; said enhancement layer includes one or more include chromium nitride (CrN), diamond-like carbon (DLC), titanium nitride (TiN), titanium oxynitride or titanium nitride oxide (TiNOₓ), zirconium nitride (ZrN), zirconium oxide (ZrO₂), zirconium oxynitride (ZrNₓO_{y}), oxyzirconium-nitrogen-carbon (ZrNC), and/or zirconium OxyCarbide (ZrOC).

7. A method for using an expandable prosthetic device comprising:
providing said expandable prosthetic device; said expandable prosthetic device comprises:
a drive block; said drive block includes a drive block opening and a slot region;
a linkage block; said linkage block includes a linkage housing and a linkage bar; said linkage bar is at least partially positioned in said linkage housing; said linkage block is movable within said slot region of said drive block;
a drive screw; said drive screw includes a head and body portion; said head is rotatably positioned in within said drive block opening; said body portion is threadingly connected to said linkage block;
first and second endplates; said first endplate includes a front portion and a rear portion; said second endplate includes a front portion and a rear portion; said rear portion of said first and second endplates are pivotally connected together; and,
first and second sets of linkages; said first set of linkage includes first and second linkages; said second set of linkage includes first and second linkages; said first linkage of said first set of linkages engages said linkage bar and said first endplate; said first linkage of said second set of linkages engages said linkage bar and said first endplate; said second linkage of said first set of linkages engages said linkage bar and said second endplate; said second linkage of said second set of linkages engages said linkage bar and said second endplate; said first and second sets of linkages are spaced from one another; and
wherein rotation of said drive screw simultaneously causes I) movement of said linkage block relative to said drive bar, and II) movement of said first endplate relative to said second endplate;
inserting said expandable prosthetic device into a patient; and
causing said drive screw to rotate which results in A) movement of said linkage block relative to said drive block and B) movement of said first endplate relative to said second endplate.

8. The method as defined in claim 7, wherein said drive screw is rotatably coupled within said drive block opening and is threadingly disposed within said linkage block.

9. The method as defined in claim 7, wherein each linkage has a first portion and a second portion that is spaced apart from said first portion along a longitudinal axis; said first portion of each of said linkages is rotatably coupled to said linkage bar and said second portion of each of said linkages engages either said first or second endplate.

10. The method as defined in claim 7, wherein at least a portion of said expandable prosthetic device includes one or more metal alloys selected from the group consisting of a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) refractory metal alloy, or l) metal alloy that includes at least 5 awt. % rhenium.

11. The method as defined in claim 7, wherein one or more of said first endplate and said second endplate includes a micro-textured surface, and/or one or more teeth.

12. The method as defined in claim 7, wherein one or more portions of said expandable prosthetic device includes an enhancement layer; said enhancement layer includes one or more include chromium nitride (CrN), diamond-like carbon (DLC), titanium nitride (TiN), titanium oxynitride or titanium nitride oxide (TiNOₓ), zirconium nitride (ZrN), zirconium oxide (ZrO₂), zirconium oxynitride (ZrNₓO_{y}), oxyzirconium-nitrogen-carbon (ZrNC), and/or zirconium OxyCarbide (ZrOC).

13. An expandable prosthetic device; said expandable prosthetic device comprises:
a drive block; said drive block includes a drive block opening;
a linkage block; said linkage block includes a linkage housing and a linkage bar; said linkage bar is at least partially positioned in said linkage housing;
a drive screw; said drive screw includes a head and body portion; said head is rotatably positioned in within said drive block opening; said body portion is threadingly connected to said linkage block;
first and second endplates; said first endplate includes a front portion and a rear portion; said second endplate includes a front portion and a rear portion; said rear portion of said first and second endplates are pivotally connected together; and,
first and second sets of linkages; said first set of linkage includes first and second linkages; said second set of linkage includes first and second linkages; said first linkage of said first set of linkages engages said linkage bar and said first endplate; said first linkage of said second set of linkages engages said linkage bar and said first endplate; said second linkage of said first set of linkages engages said linkage bar and said second endplate; said second linkage of said second set of linkages engages said linkage bar and said second endplate; said first and second sets of linkages are spaced from one another; and
wherein rotation of said drive screw causes I) movement of said linkage block relative to said first and/or second end plates along a longitudinal length of said expandable prosthetic device when said expandable prosthetic device moves from a fully closed or collapsed position to a fully open or expanded position, and II) movement of said first endplate relative to said second endplate when said expandable prosthetic device moves from said fully closed or collapsed position to said fully open or expanded position; and
wherein said drive block and linkage block are spaced from one another when said expandable prosthetic device is in said fully closed or collapsed position, and a spacing between said drive block and linkage block is reduced as said expandable prosthetic device moves from said fully closed or collapsed position to said fully open or expanded position.

14. The expandable prosthetic device as defined in claim 13, wherein said drive block moves along a longitudinal length of said expandable prosthetic device as said expandable prosthetic device moves from said fully closed or collapsed position to said fully open or expanded position.

15. The expandable prosthetic device as defined in claim 13, wherein a front or proximal plane of said linkage block remains in a same plane as a front or proximal edge of said first and second endplates as said expandable prosthetic device moves from said fully closed or collapsed position to said fully open or expanded position.

16. The expandable prosthetic device as defined in claim 13, wherein a distal end portion of said linkage block includes a slotted region that enables movement of said linkage block along said longitudinal length of said expandable prosthetic device as said expandable prosthetic device moves from said fully closed or collapsed position to said fully open or expanded position.

17. The expandable prosthetic device as defined in claim 16, wherein a connection arrangement connects said slotted region of said linkage block to a rear region of said first and/or second endplates.

18. The expandable prosthetic device as defined in claim 13, wherein said drive screw is rotatably coupled within said drive block opening and is threadingly disposed within said linkage block.

19. The expandable prosthetic device as defined in claim 13, wherein each linkage has a first portion and a second portion that is spaced apart from said first portion along a longitudinal axis; said first portion of each of said linkages is rotatably coupled to said linkage block and said second portion of each of said linkages engages either said first or second endplate.

20. The expandable prosthetic device as defined in claim 13, wherein at least a portion of said expandable prosthetic device includes one or more metal alloys selected from the group consisting of a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) refractory metal alloy, or l) metal alloy that includes at least 5 awt. % rhenium.

21. The expandable prosthetic device as defined in claim 13, wherein one or more of said first endplate and said second endplate includes a micro-textured surface, and/or one or more teeth.

22. The expandable prosthetic device as defined in claim 13, wherein one or more portions of said expandable prosthetic device includes an enhancement layer; said enhancement layer includes one or more include chromium nitride (CrN), diamond-like carbon (DLC), titanium nitride (TiN), titanium oxynitride or titanium nitride oxide (TiNOₓ), zirconium nitride (ZrN), zirconium oxide (ZrO₂), zirconium oxynitride (ZrNₓO_{y}), oxyzirconium-nitrogen-carbon (ZrNC), and/or zirconium OxyCarbide (ZrOC).

23. A method for using an expandable prosthetic device comprising:
providing said expandable prosthetic device; said expandable prosthetic device comprises:
a drive block; said drive block includes a drive block opening;
a linkage block; said linkage block includes a linkage housing and a linkage bar; said linkage bar is at least partially positioned in said linkage housing;
a drive screw; said drive screw includes a head and body portion; said head is rotatably positioned in within said drive block opening; said body portion is threadingly connected to said linkage block;
first and second endplates; said first endplate includes a front portion and a rear portion; said second endplate includes a front portion and a rear portion; said rear portion of said first and second endplates are pivotally connected together; and,
first and second sets of linkages; said first set of linkage includes first and second linkages; said second set of linkage includes first and second linkages; said first linkage of said first set of linkages engages said linkage bar and said first endplate; said first linkage of said second set of linkages engages said linkage bar and said first endplate; said second linkage of said first set of linkages engages said linkage bar and said second endplate; said second linkage of said second set of linkages engages said linkage bar and said second endplate; said first and second sets of linkages are spaced from one another; and
wherein rotation of said drive screw causes I) movement of said linkage block relative to said first and/or second end plates along a longitudinal length of said expandable prosthetic device when said expandable prosthetic device moves from a fully closed or collapsed position to a fully open or expanded position, and II) movement of said first endplate relative to said second endplate when said expandable prosthetic device moves from said fully closed or collapsed position to said fully open or expanded position; and
wherein said drive block and linkage block are spaced from one another when said expandable prosthetic device is in said fully closed or collapsed position, and a spacing between said drive block and linkage block is reduced as said expandable prosthetic device moves from said fully closed or collapsed position to said fully open or expanded position;
inserting said expandable prosthetic device into a patient; and
causing said drive screw to rotate which results in A) movement of said linkage block relative to said drive block and B) movement of said first endplate relative to said second endplate.

24. The method as defined in claim 23 wherein said drive block moves along a longitudinal length of said expandable prosthetic device as said expandable prosthetic device moves from said fully closed or collapsed position to said fully open or expanded position.

25. The method as defined in claim 23, wherein a front or proximal plane of said linkage block remains in a same plane as a front or proximal edge of said first and second endplates as said expandable prosthetic device moves from said fully closed or collapsed position to said fully open or expanded position.

26. The method as defined in claim 23, wherein a distal end portion of said linkage block includes a slotted region that enables movement of said linkage block along said longitudinal length of said expandable prosthetic device as said expandable prosthetic device moves from said fully closed or collapsed position to said fully open or expanded position.

27. The method as defined in claim 26, wherein a connection arrangement connects said slotted region of said linkage block to a rear region of said first and/or second endplates.

28. The method as defined in claim 23, wherein said drive screw is rotatably coupled within said drive block opening and is threadingly disposed within said linkage block.

29. The method as defined in claim 23, wherein each linkage has a first portion and a second portion that is spaced apart from said first portion along a longitudinal axis; said first portion of each of said linkages is rotatably coupled to said linkage block and said second portion of each of said linkages engages either said first or second endplate.

30. The method as defined in claim 23, wherein at least a portion of said expandable prosthetic device includes one or more metal alloys selected from the group consisting of a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) refractory metal alloy, or l) metal alloy that includes at least 5 awt. % rhenium.

31. The method as defined in claim 23, wherein one or more of said first endplate and said second endplate includes a micro-textured surface, and/or one or more teeth.

32. The method as defined in claim 23, wherein one or more portions of said expandable prosthetic device includes an enhancement layer; said enhancement layer includes one or more include chromium nitride (CrN), diamond-like carbon (DLC), titanium nitride (TiN), titanium oxynitride or titanium nitride oxide (TiNOₓ), zirconium nitride (ZrN), zirconium oxide (ZrO₂), zirconium oxynitride (ZrNₓO_{y}), oxyzirconium-nitrogen-carbon (ZrNC), and/or zirconium OxyCarbide (ZrOC).
